(19) United States
(12) Patent Application Publication
(10) Pub. No.: US 2022/0331576 A1
(43) Pub. Date: Oct. 20, 2022

(54) CIRCULATORY ASSIST PUMP

(71) Applicant: Second Heart Assist, Inc., Salt Lake City, UT (US)

(72) Inventor: Howard J. Leonhardt, Mission Viejo, CA (US)

(21) Appl. No.: 17/698,287

(22) Filed: Mar. 18, 2022

Related U.S. Application Data
(63) Continuation-in-part of application No. 17/098,162, filed on Nov. 13, 2020.
Application No. 17/098,162 is a continuation-in-part of application No. 16/982,908, filed on Sep. 21, 2020.
(60) Provisional application No. 63/263,133, filed on Oct. 27, 2021.
(60) Provisional application No. 62/645,599, filed on Mar. 20, 2018.
(60) Provisional application No. 62/682,046, filed on Jun. 7, 2018.
(60) Provisional application No. 62/694,564, filed on Jul. 6, 2018.

(51) Int. Cl. A61M 60/13; A61M 60/139; A61M 60/422; A61M 60/523; A61M 60/569; A61M 60/808

(52) U.S. Cl. CPC A61M 60/13; A61M 60/139; A61M 60/422; A61M 60/523; A61M 60/569; A61M 60/808; A61M 2205/0266; A61M 2205/3523; A61M 2205/8262; A61M 2205/3365

(57) ABSTRACT

A minimally invasive circulatory support platform that utilizes an aortic stent pump or pumps. The platform uses a low profile catheter-based techniques and provides temporary and chronic circulatory support depending on the needs of the patient. Further described is a wirelessly powered circulatory assist pump for providing chronic circulatory support to, for example, heart failure patients. The platform and system are relatively easy to place, have higher flow rates than existing systems, and provide improvements in the patient's renal function.

### CROSS-REFERENCE TO RELATED APPLICATIONS

This application claims the benefit of U.S. Provisional Patent Application Ser. No. 63/263,133, filed on Oct. 27, 2021; this application is also a continuation-in-part application of U.S. patent application Ser. No. 17/098,162, filed Nov. 13, 2020, which is a continuation-in-part application of U.S. patent application Ser. No. 16/982,908, filed Sep. 21, 2020, which is the national phase entry under 35 U.S.C. § 371 of International Patent Application PCT/US2019/023208, filed Mar. 20, 2019, designating the United States of America and published as International Patent Publication WO 2019/183247 A1 on Sep. 26, 2019, which claims the benefit of U.S. Provisional Patent Application Ser. No. 62/645,599, filed Mar. 20, 2018; U.S. Provisional Patent Application Ser. No. 62/682,046, filed Jun. 7, 2018; and U.S. Provisional Patent Application Ser. No. 62/694,564, filed Jul. 6, 2018, the contents of the entirety of each of which are hereby incorporated herein by this reference.

### TECHNICAL FIELD

The application relates generally to medical devices, and more particularly to a system, apparatus, and associated methods for assisting a subject's heart to pump blood (e.g., a circulatory assist pump).

### BACKGROUND

U.S. Pat. No. 8,617,239 to Reitan (Dec. 13, 2013), the contents of which are incorporated herein by this reference, relates to a catheter pump to be positioned in the ascending aorta near the aortic valve of a human being, comprising an elongated sleeve with a drive cable extending through the sleeve and connectable at its proximal end to an external drive source and a drive rotor near the distal end of the drive cable mounted on a drive shaft being connected with the drive cable. The drive rotor consists of a propeller enclosed in a cage and the propeller and the cage are foldable from an insertion position close to the drive shaft to an expanded working position, which are characterized by means for anchoring the drive rotor in the ascending aorta near the aortic valve after insertion. Also described is a method to position the pump of a catheter pump in the ascending aorta just above the aortic valve.

U.S. Pat. No. 8,617,239 to Reitan builds upon an earlier patent of Reitan, i.e., U.S. Pat. No. 5,749,855 to Reitan (May 12, 1998), the contents of which are also incorporated herein by this reference, which relates to a drive cable, with one end of the drive cable being connectable to a drive source and a collapsible drive propeller at the other end of the drive cable. The collapsible drive propeller is adjustable between a closed configuration in which the collapsible drive propeller is collapsed on the drive cable and an open configuration in which the collapsible drive propeller is expanded so as to be operative as an impeller. A sleeve extends between one side of the collapsible drive propeller and the other side of the collapsible drive propeller with the sleeve being movable between configurations in which the collapsible drive propeller is in the open and closed configuration. A lattice cage is arranged surrounding the propeller and is folded out at the same time as the propeller.

As described by U.S. Pat. No. 8,617,239 to Reitan, while the device of U.S. Pat. No. 5,749,855 operates very well in many circumstances, there is still room for improvement. For example, it would be safer if the lattice cage folded out before the propeller folded out. In addition, the shaft supporting the propeller needs to be journaled with bearings, and such bearings require lubrication.

An even earlier blood pumping catheter is described in U.S. Pat. No. 4,753,221 to Kensey et al. (Jun. 28, 1988), the contents of which are incorporated herein by this reference. Kensey et al. relates to an elongated catheter for pumping blood through at least a portion of a subject's vascular system. The catheter is of a sufficiently small diameter and flexibility to enable it to be passed through the vascular system so that the distal end portion of the catheter is located within or adjacent the patient's heart. A rotatable pump is located at the distal end of the catheter and is rotated by drive means in the catheter. The distal end portion of the catheter includes an inlet for blood to flow therein and an outlet for blood to flow therefrom. The catheter is arranged so that blood is pumped by the catheter's pump through the heart and into the vascular system without requiring any pumping action of the heart.

Other catheter pumps are known from US 2008/0132748 A1, US 2008/0114339 A1, and WO03/103745A2, the contents of each of which are incorporated herein by this reference.

### BRIEF SUMMARY

Described, among other things herein, is a minimally invasive circulatory support platform that utilizes an aortic stent pump. The platform uses a low profile, catheter-based technique and can be used to provide temporary and chronic circulatory support depending on the needs of the subject or patient (e.g., a mammal, such as a human).

In certain embodiments, the described device includes a temporary circulatory assist pump on the tip of an aortic catheter.

In certain embodiments, the device includes a further pump placed intermediate between the catheter tip and herein described handle for placement of the further pump in the aorta, right above the renal arteries.

In certain embodiments, the described device includes a wireless powered circulatory assist pump (or pumps) positioned within an aortic stent.

In certain embodiments, the described device includes a battery powered circulatory assist pump (or pumps) positioned within an aortic stent which may be wirelessly charged.

Also described is a catheter-based, temporary circulatory assist pump (e.g., powered by an associated endovascular catheter with a drive) for use in treating a patient with acute decompensated heart failure, which pump provides circulatory support to a subject undergoing high risk percutaneous coronary intervention (“PCI”). Such a temporary circulatory support pump is typically placed within an aortic stent on the tip of a catheter placed just above the renal arteries in the descending aorta. The catheter is of sufficiently small diameter and flexibility to enable it to be passed through the vascular system so that the distal end portion of the catheter can be appropriately placed within the aorta. This reduces workload on a patient's heart, and improves lower extremity perfusion.

When the catheter is disconnected from the stent after placement in the aorta, the stent can be switched to wireless power. The wireless electromagnetic power communicates directly with, e.g., iron filled (+) and (−) polarized tips of impeller blades. The pump may be combined with a removable wireless powered pulsatile mesh stent, which is placed above the catheter higher in the aorta. QUT repeater technology may be included for enhanced wireless power. “Wireless system to power heart pumps could save lives currently lost to infection,” (May 15, 2017, Queensland University of Technology), https://phys.org/news/2017-05-wireless-power-heart-lost-infection.html, the contents of which are incorporated herein by this reference.

Further described is a wirelessly powered circulatory assist pump (an aortic stent implant) that provides chronic circulatory support for heart failure patients. A wireless powered chronic implant can be removable and can utilize both continuous and pulsatile flow.

The described platform and system are relatively easy to place, have higher flow rates than existing systems, and provide improvements in a patient's renal function. The chronic circulatory assist device (which is removable) is placed within an aortic stent that is preferably wirelessly powered, and combined with, e.g., a vibrational harmonic energy technology or electric charge surface treatment to reduce or prevent blood clot (thrombus) formation, which may be associated with the device. Such a system features both a rotating impeller within a lower positioned aortic stent and a pulsating cuff aortic stent, which is placed above the primary stent pump. The impeller is shaped and designed to maximize safety and blood flow and to reduce the risk of hemolysis. Also described is a low RPM impeller system that displays higher flow, less heat, and less hemolysis risk for the patient.

Further described is a platform that may be used to provide circulatory assist support by maximizing cardio and renal function recovery, while at the same time minimizing risk of thrombosis, stroke, hemolysis, mechanical breakdown, infection, and heart valve damage. Further, because the impeller is positioned relatively far from the heart (e.g., just above the renal arteries in the aorta, see Int. J. Card. 2018; 275 (2019)53-58), the natural pulsatility of a heart beat is maintained. The impeller simply works in cooperation and harmony with the pulse waves. In contrast, prior art placement within or near the heart interferes with natural pulsatility. Preferably, flow and energy use are optimized via timing of pulsations and impeller turn speeds with natural heart pulsatile flow.

The system or “loop” may be automatically read and adjusted to maximize power usage, battery life, long term durability, flow, and patient blood pressure(s) that self-adjusts automatically in response to changing conditions of the patient such as sleep and exercise.

Particularly described is, e.g., a catheter-based circulatory assist pump and methods of using it. Such a pump assists the subject's heart's pump function. The circulatory assist pump is primarily intended for use in assisting a subject suffering from heart failure.

Also particularly described is a circulatory assist pump intended for implantation that comprises a tubular elongated casing, which is associated with a plurality of impellers, which fold and extend therefrom, through which a shaft passes. The shaft has means (e.g., an actuation cable and/or associated cam system) that extends the impeller arm-like blades perpendicularly and preferably also retracts them. Because of the outwardly-foldable arm-like impeller blades, the catheter can be made very narrow, which is advantageous during introduction or implantation into the subject's circulatory system, but nevertheless provides a powerful flow effect when the blades are in their extended condition.

In use, the catheter may be introduced “percutaneously” into the lower aorta via, e.g., the normal “Seldinger technique” in the groin (a small incision into the femoral artery) and fed up to the aorta to the desired position (e.g., the descending aorta). The pump may be inserted in the groin area and introduced into the femoral artery (e.g., to just above the renal arteries in the descending aorta) with the help of a small surgical insertion and insertion sheath. The pump is thereafter fed up into the desired position in the lower aorta.

Alternatively, the pump may be placed via axillary entry in the neck or chest of the subject. See, e.g., K M. Doersch “Temporary Left Ventricular Assist Device Through an Axillary Access is a Promising Approach to Improve Outcomes in Refractory Cardiogenic Shock Patients,” ASAIO J. 2015 May-June; 61(3): 253-258; doi: 10.1097/MAT.0000000000000222, the contents of which are incorporated herein by this reference, which describes implantation of a temporary left ventricular assist device (“LVAD”) through an axillary approach as a way to provide adequate circulation to the patient, avoid multiple chest entries and infection risks.

Treatment will typically continue for six (6) hours, but may last, for example, for 72 hours.

A preferred embodiment utilizes a monorail guidewire lumen “rapid exchange” (“RX”) system, where the guidewire lumen may extend proximally only. See, e.g., US 2003/0171642 A1 to Schock et al. (Sep. 11, 2003) and J. Schroeder 2013 Peripheral Vascular Interventions: An Illustrated Manual, “Balloon Catheters Over the Wire and Monorail,” DOI: 10.1055/b-0034-65946, the contents of each of which are incorporated herein by this reference.

In order to avoid the impeller damaging the surrounding tissue, the pump is preferably encased within a cage, stent, or “stent cage” that shields, e.g., the subject's aortic tissue from the impeller. The (aortic) stent cage preferably has a highly open flow. It is sized and made of a material that provides for stability against the aortic wall of the subject, where it is preferably strongly affixed to the aortic wall. Preferably, the aortic stent cage has just the right radial force to distend the aorta, for example, two (2) mm, giving extra flow and a safety area and which stabilizes position of pump securely within the subject's aorta. Accordingly, an aortic stent cage that is positionally stable may offer certain advantages compared to other systems like PROCYRION™ that reportedly migrate up the subject's aorta with the motor “on” and down the subject's aorta with the motor “off”. Systems like PROCYRION™ that include a gap between the aortic stent/protective cage and the aorta wall allows for back and forth motion, which increases turbulence of flow and increasing the risk of dislodging thrombus from the aortic wall, and causing much more damage than secure fixation. Furthermore, flow thrust is lost when the tip of the aortic stent pump bounces back and forth in the aorta, which is reduced with the instant design.

A wireless drive is preferably utilized to drive the pump. Such a drive is typically in the form of an external power belt (electric powered copper coil inside) and appropriate circuitry that fits around the patient's abdomen, which belt provides a magnetic field that drives and/or controls rotation of the impeller.

In some embodiments, a battery and motor are utilized to drive the pump. An external power belt, or other external device, may be provided that wirelessly charges the battery.

The impeller blades' tips preferably comprise a material subject to magnetic forces. The impeller can also be provided with an elastic rubber sheath (not shown) which reduces tissue damage and which can also increase the pressure effect.

In further embodiments, the impeller blades may be configured to change shape on demand.

In certain embodiments, sensors are used with the system, e.g., to monitor hemolysis and/or impeller speed, and the pulsations of cuffs are adjusted as desired to balance a minimization of hemolysis with a maximization of flow utilizing the system.

In certain embodiments, a pulsating stent graft in the patient's upper aorta and an impeller turning circulatory assist pump placed in a bare aortic stent in the patient's lower aorta may be used in combination, with timing optimized. For instance, appropriately placed sensors may be used to optimize the timing of pulsations of the upper aortic stent graft and the revolutions per minute of a lower bare aortic stent impeller circulatory assist pump.

In order to avoid thrombo-embolismic complications, the circulatory assist pump or parts thereof can be, e.g., heparinized.

The actuation cable can be in the form of a compact cable that runs through the tubular elongated casing of the catheter. The actuation cable has such a construction that the impeller folds outwardly with forward movement of the actuation cable by the physician.

The tubular elongated casing can be surrounded by a sleeve or a tube of an elastic material such as rubber or similar.

In its extended condition, the impeller preferably has a working diameter about 23 mm for an adult human.

In practice, the described system may be used to not only sustain a (e.g., congestive heart failure) patient's life, but also may be used to provide mechanical circulatory assistance for, e.g., up to 36 months, during the course of heart rehabilitation/regeneration treatment.

The described system offers advantages over existing heart assist devices in that it need not cross the aortic valve, and location positioning of the device is not as strict as with existing devices, meaning there is less need to reposition the device. Furthermore, the system maintains arterial pulsatility, does not require a high pump speed (e.g., 7,500 vs. 33,000 rpm), reduces hemolysis, and reduces acute kidney injury.

### BRIEF DESCRIPTION OF THE DRAWINGS

FIG. 1 depicts a lobe design according to the instant disclosure displaying deployed (or extended) arm-like impeller blades.

FIG. 2 depicts the lobe design of FIG. 1 displaying retracted arm-like impeller blades.

FIG. 3 depicts a front view of the lobe design of FIG. 2 displaying deployed (operational) impellers.

FIG. 4 is a cross-sectional view of the device of FIG. 1.

FIGS. 5 and 6 show an impeller blade's shape.

FIG. 7 depicts a stent cage, at the tip of the catheter, which surrounds the arm-like impeller blades, where, e.g., a wirelessly driven impeller is contained within a protective cage stent.

FIG. 8 depicts the prior art pulsatile stent of the incorporated herein Palma et al.

FIG. 9 is a picture of a device as described herein connected to a drive axis placed within a pig.

FIG. 10 shows an overall schematic of a system according to the disclosure (not to scale).

FIG. 11 shows a more detailed view of an alternative embodiment of the device.

FIG. 12 depicts a belt and controller positioned on a human subject.

FIG. 13 depicts a physiologically accurate mock circulation loop.

FIG. 14 depicts a “Biomerics Advanced Catheter” having a catheter, catheter connector, drive shaft, handle, impeller, stent cage, and tip.

FIG. 15 depicts a front view of the stent cage of FIG. 7 showing the highly open design of the cage.

FIG. 16 depicts an alternative embodiment of a stent cage, at the tip of a catheter, which is to surround the rotating impeller blades of the circulatory assist pump.

FIG. 17 depicts a catheter with deployed impellers encaged within the stent cage at the tip of the catheter to the right of its associated cross-sectional view taken along lines A-A.

FIG. 18 depicts a wireless circulatory assist pump according to an embodiment of the present disclosure.

FIG. 19 depicts an impeller according to an embodiment of the present disclosure.

FIG. 20A depicts a blade of the impeller of FIG. 19 in an initial state.

FIG. 20B depicts the blade of FIG. 20A in an extended state.

FIG. 21 depicts a circulatory assist system including a wireless circulatory assist pump in an expanded state, according to embodiments of the present disclosure.

FIG. 22 depicts the wireless circulatory assist system of FIG. 21 including a wireless circulatory assist pump in a collapsed state, according to embodiments of the present disclosure.

FIG. 23A depicts impeller blades of the wireless circulatory assist pump of FIG. 21.

FIG. 23B depicts impeller blades of a wireless circulatory assist pump, according to additional embodiments of the present disclosure.

FIG. 24 depicts a wireless circulatory assist system including a wireless circulatory assist pump in an expanded state, according to embodiments of the present disclosure.

### DETAILED DESCRIPTION

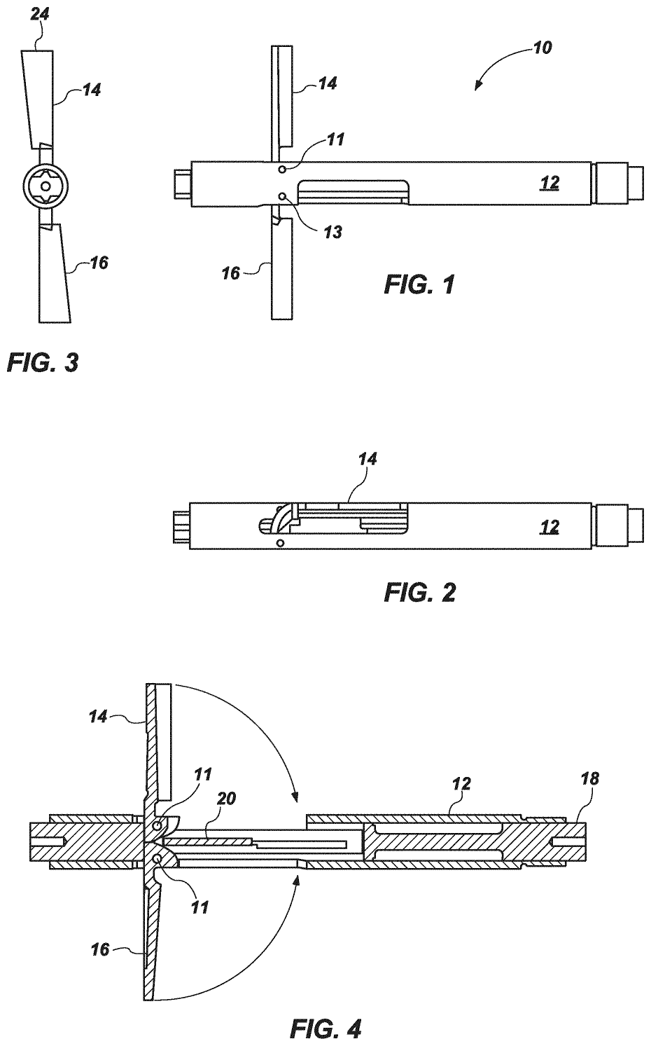

An aspect of the disclosure is a circulatory assist pump, generally 10, shown in FIGS. 1, 3, and 4 in its operational position. The circulatory assist pump 10 comprises a tubular elongated casing 12 associated with a pair of arm-like impeller blades 14, 16. The depicted impeller blades are pivotally associated with the remainder of the lobe by pivots (e.g., pins or shafts) 11 placed in apertures 13 in the tubular elongated casing 12 and impeller blades. The impeller blades are outwardly foldable and retractable, and can move, e.g., into a position perpendicular to the tubular elongated casing 12. As can be determined, the accompanying figure drawings are generally not drawn to scale.

The depicted circulatory assist pump includes a positioning cable 18 running along the impeller axis, about which the impeller blades 14, 16 (along with the rest of the device) rotate to create a pump action, for example, in the aorta. The arm-like nature of the depicted blades allows them to extend maximally from the remainder of the body when in a perpendicular position and fill a large portion of the descending aorta. At the end of the positioning cable is a rod 20 that interacts with a cam portion 22 of each impeller blade (see, e.g., FIGS. 4 and 6). Advancing (or relatively displacing) the rod 20 so that it abuts and actuates the cam portion 22 causes the withdrawn impeller (FIG. 3) to extend outwards from the rest of the lobe (FIG. 1). The cam lobe design (FIG. 4) is utilized to expand and retrieve the impeller into and out of the catheter, which is far more reliable deployment than with, for example, a spring design, although a spring may also be used herein. For example, springs vary with temperature and manufacturing, while cam lobes are consistent and remain constant.

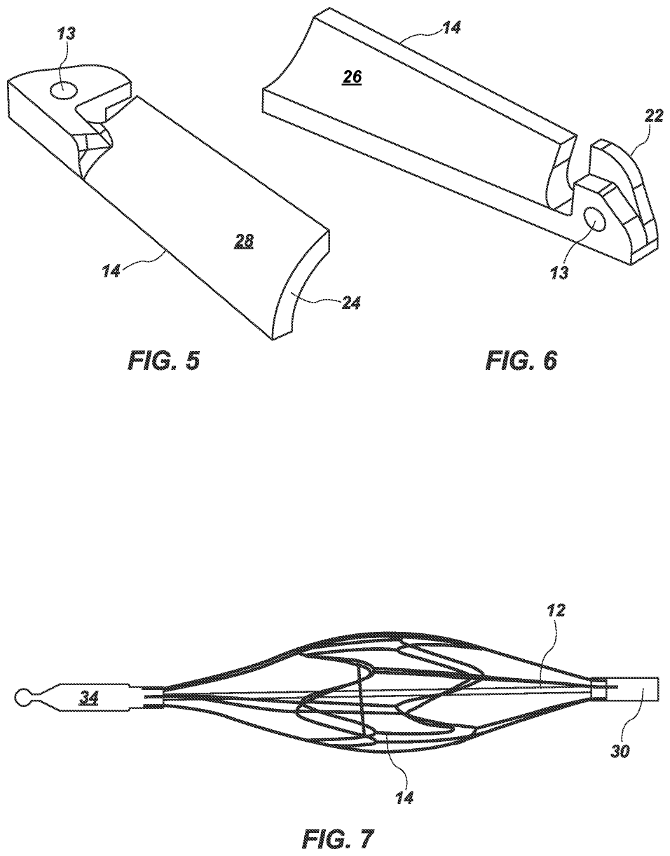

As depicted in FIGS. 5 and 6, each impeller blade has a tip 24, face 26, and back 28 (any or all of which may be magnetic so as to be driven by a wireless drive). The impeller shape design as depicted in FIGS. 5 and 6 maximizes blood flow at low power/lower RPMs, while reducing hemolysis and heat. Lower RPMs mean less power needs, improving a system powered wirelessly. There is also reduced risk of a mechanical breakdown. Materials that can be magnetized, which are also the ones that are strongly attracted to a magnet, are called ferromagnetic (or “ferrimagnetic”). Such materials include iron, nickel, cobalt, some alloys of rare-earth metals, and some naturally occurring minerals.

In certain embodiments, the impeller blades can be tilted on demand (in the same manner as the way an airplane wing flaps are controlled) by, e.g., adjustment of the cams, which balances hemolysis, thrust, and flow; maximizes flow with a temporary increase in hemolysis; and can be used to catch native aortic flow to re-charge a battery in the center spindle.

An aortic stent cage 32 surrounds the impeller (see, e.g., FIGS. 7, 11, and 14-17) and preferably has the most open area possible (see, e.g., FIG. 15), so as to reduce hemolysis. The system thus matches greater strength and protection in balance. The wire-like elements of the stent cage 32 are preferably rounded and are not too thin (like razor wire that can cut blood cells) or too thick like the prior art's flat elements, which can smack hard against and damage blood cells (hemolysis) on their flat surface planes. The depicted aortic stent protective cage 32 with high flow through areas has rounded elements and balance stability strength with low hemolysis and high flow. Preferably, the aortic stent has strength and not too many flat cage elements to damage blood cells and inhibit flow. This may be achieved by use of the rounded cage elements and by design permitting high radial force and strength (certain prior art devices do not even reach the aortic wall (e.g., <20 mm in an adult human) and bounce back and forth in large aortas).

Prior art devices have been known to migrate up and down and bounce side to side in the aorta. Their flow is disturbed and energy is lost in the process. Their movement causes turbulence, which promotes blood clotting and hemolysis.

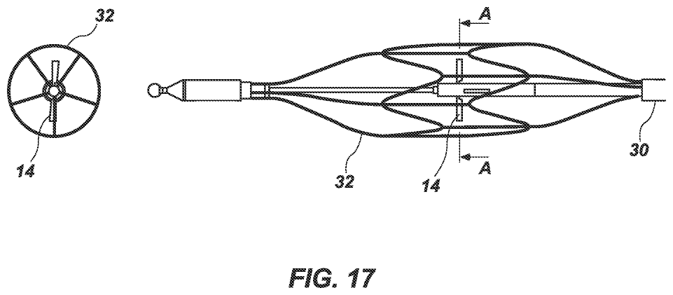

An aortic stent as described herein (see, e.g., FIG. 17) can be detached from the associated drive shaft and external motor controller (which are removed from the patient) and can be converted to wireless power. For example, instead of being driven by the drive axis, the pump can then be powered via, e.g., an external belt system or wireless power WiFi in the patient's home or workplace.

The system is preferably positioned and stabilized in the aorta and the available impeller space is widened with a high radial force aortic stent that distends the aortic wall inner diameter, for example, about two (2) mm. Such positioning allows more flow and more use of the entire area of the aorta, particularly in comparison to the prior art. Such aortic stent strength stabilizes position and reduces the need for repositioning.

In preferred embodiments, a confirming high radial force aortic stent provides for firm stability of fixation of position without the need for hooks. Such a system distends the diameter of the aorta by about two (2) mm (on average), which provides more space available for impeller use.

The expandable stent may be manufactured and adapted for use herein in accordance with techniques known by those of skill in the art (see, e.g., U.S. Pat. No. 5,354,308 to Simon et al. (Oct. 11, 1994), U.S. Pat. No. 4,580,568 to Cesare Gianturco (Apr. 8, 1986), and U.S. Pat. No. 5,957,949 to Leonhardt et al. (Sep. 28, 1999), the contents of each of which are incorporated herein by this reference).

Depicted is a circulatory assist pump within a bare aortic stent at the tip of a 13.8 French (“FR”) catheter for temporary support. The aortic stent with impeller (e.g., FIG. 7) may be driven by a drive line associated with the placement catheter 30, or disconnected from the catheter and switched to wireless power. In the embodiment of FIG. 7, there is a simple impeller in the stent cage on tip 34 of the catheter with vibrational energy delivered via the drive shaft.

In certain embodiments, the catheter protective cage aortic stent expands and compresses easily, e.g., to pass another catheter by the stent cage. For example, a standard PCI catheter was run up the outside of the stent cage and was of no issue. The radial force of the stent is insufficient to collapse the PCI catheter, particularly when placed against a compliant aorta. The stent typically presses the PCI catheter about 1 mm into the aorta wall and leaves open the whole aorta for the impeller with a large safety gap. The impeller may be angled down like arrow feathers, and then there is even more room for placing a PCI catheter.

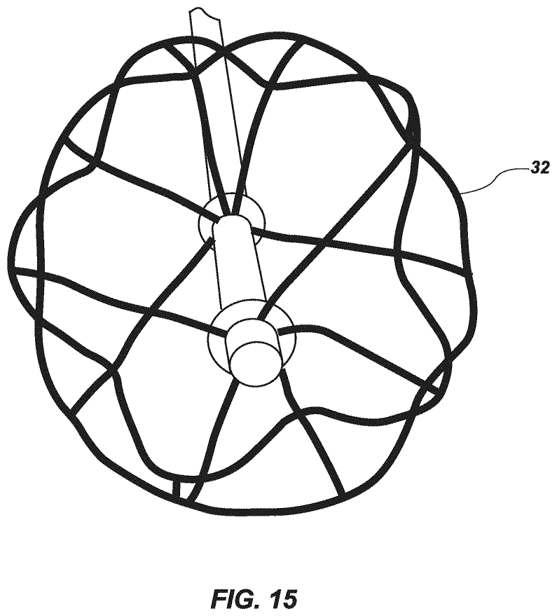

The protective cage opens and closes relatively easily with a simple turn of the wheel on a handle associated with the catheter (FIG. 14). Collapsing it partially (or fully) allows for the passage of the PCI catheter and then may be opened up fully when the PCI catheter is in place.

As best depicted in FIG. 15 (a front view of the stent cage 32), the (aortic) stent cage 32 is preferably designed with a highly open flow to prevent damage to, e.g., the patient's blood cells, such as hemolysis and also reduces the risk of thrombosis.

In certain embodiments (e.g., to reduce the chance that the impeller impacts the stent cage 32 on the side where the PCI catheter is present), the impeller is not extended all of the way (e.g., instead of opening it 11.5 mm wide in a 22 mm aorta, it is only opened, e.g., 8 mm wide, but it still provides 80 to 90% of the flow as compared to when the impeller blades are fully open).

In certain embodiments, the impeller is first started turning with the blades, e.g., only half way open, and after it has been confirmed (e.g., either by measuring flow, viewing the situation, or otherwise) that sufficient gap space exists in the aorta, then the impeller is, e.g., fully opened. This serves to allow one to pump in smaller aortas. A half open impeller diameter is only about 8 mm, while fully open may be, e.g., 11.5 to 18 mm depending on size. Only about 20% of the flow is lost at “half open” in comparison to full open. In some test cases, the flow at “half open” was equal to the flow at full open in animal studies at Tufts Medical Center.

In certain embodiments, magnetized impeller blade tips may be powered wirelessly by an external power belt (electrically powered with a copper coil inside) place around, e.g., the patient's abdomen. Wireless power enables the system to provide the patient with a better quality of life, while reducing the risk of infections and providing the physician with greater patient management options. Wireless power systems are disclosed in, e.g., J. Bowler “This Wireless Heart Pump Battery Could Save Thousands of Lives” ScienceAlert (May 26, 2017) and Knecht et al., “High Efficiency Transcutaneous Energy Transfer for Implantable Mechanical Heart Support Systems” (November 2015); DOI: 10.1109/TPEL.2015.2396194, the contents of each of which are incorporated herein by this reference. Such a transcutaneous energy transfer system (“TETS”) may be used, e.g., with a ventricular assist device. A TETS system setup includes a power converter, rectifier, and coils. See, also, Ho et al. “Midfield Wireless Powering for Implantable Systems,” Proceedings of the IEEE, pp. 1-10 (2013 IEEE), the contents of which are also incorporated herein by this reference.

In certain embodiments, WiFi power may be used to control and power the device/system (with WiFi power) instead of using a belt. In such embodiments, repeater, booster, and/or extender technology, may be used with an external wireless power belt to reduce irritation and heating of, e.g., the subject's skin. See, e.g., “WiFi Boosters, Repeaters and Extenders” RepeaterStore, (https://www.repeaterstore.com/pages/wifi-booster-repeater-extender-differences) (accessed Feb. 26, 2018), the contents of which are incorporated herein by this reference. The system preferably utilizes wireless repeater power with minimal skin irritation. See, also, D. Gershgorn “Your Wireless Internet Could Power Your Future Devices” Popular Science, (popsci.com/your-wireless-internet-could-power-your-future-devices) (Jun. 3, 2015) and J. Langston “Popular Science names ‘Power Over Wi-Fi’ one of the year's game-changing technologies,” UW News, (washington.edu/news/2015/11/18/popular-science-names-power-over-wi-fi-one-of-the-years-game-changing-technologies/) (Nov. 18, 2015), the contents of each of which are incorporated herein by this reference.

Wireless control of the system can also be used to promote expression of desirable protein(s) via, e.g., implanted micro coils on the stent. See, e.g., US Patent publication US 2017/0266371 A1 to Leonhardt et al. (Sep. 21, 2017), the contents of which are incorporated herein by this reference, for protein expression signals. These micro coils too can utilize wireless energy. Wireless control can extend to pulsatility, speed, and/or impeller angle of the various components of the system. The micro coils can be utilized to control release and/or expression of protein(s) in the aorta, including the release and/or expression of elastin to improve the elasticity of the aorta and mediate stem cell homing and the release and/or expression of follistatin to build new, strong, thick smooth muscle.

The pump may be placed, for example, above the renal arteries in the aorta to aid in kidney function. More flow into the kidneys means more rapid removal of excess fluids, which leads to better revival of kidney function. In certain embodiments, the system preferably uses the full diameter of the aorta to increase pump stability and reduce pump migration.

In animal studies using the described system in sheep and swine, 1.5 to 2.0 liters of true augmented blood (beyond native cardiac output) were provided. With direct flow cannulas placed into the kidneys, the system able to augment renal blood flow by 25 to 50%. The pump was able to generate a gradient of more than 10 mm to unload the left ventricle and achieve improved hemodynamics without any clinically significant steal (reversed flow in the artery). Further, there was a reduced cardiac work index. There was also a significant increase in urine output and no significant hemolysis.

Indications for use of the described system include cardio-renal syndrome, protecting renal function during PCI, and chronic heart failure.

The outwardly foldable impeller uses rotational motion to draw blood in and down from the heart, and moves the blood down the aorta while itself remaining stationary due to the positioning of the cage stent within the aorta. In certain embodiments, controls (e.g., wireless controls) are utilized to modify the rotating impeller blade angles in order to, for example, change flow characteristics. This can be used, e.g., in short durations to dramatically increase flow at the expense of temporary increase of hemolysis, but the system can revert back to a low hemolysis angle shortly thereafter.

The impeller maximizes blood flow, while minimizing hemolysis, power needs, RPMs, and turbulence. The system preferably uses the least RPMs and highest flow and thus lowest hemolysis. The use of a simple impeller lowers the risk of mechanical failure.

Wireless technology can also be used to re-charge a battery or back up a battery for the system as needed.

In one embodiment (not shown), a battery backup power source is housed in the center spindle of the circulatory assist pump, which battery backup power source can be charged either by impeller blade turns or by wireless external recharging.

In certain embodiments, wireless power also powers the turns of the magnetized impeller blades directly, and battery power is only used as a backup.

In certain embodiments, the system includes implanted sensors that assist with a real time, automatic adjustment and management of the circulatory assist support system based upon data provided by the implanted (preferably wireless) sensors. The sensors monitor fluid flow and provide feedback and data to the system, which feedback and data is used to, e.g., adjust the speed and/or angle of the impeller to increase or decrease fluid flow and pressure.

Sensor(s) monitor hemolysis levels and automatically adjust the balance of RPM speed of the impellers and the pulsations of the cuffs (if present), to balance the minimization of hemolysis with the maximization of flow efficiency.

In certain embodiments, the system includes means for synchronous pumping, which is determined by the sensors. See, e.g., Gohean et al. “Preservation Of Native Aortic Valve Flow And Full Hemodynamic Support With The TORVAD™ Using A Computational Model Of The Cardiovascular System,” ASAIO J. 2015 May-June; 61(3): 259-265; doi: 10.1097/MAT.0000000000000190, the contents of which are incorporated herein by this reference.

The range of blood flow parameters in the ascending aorta that can result from various angulations of outflow graft anastomosis of a left ventricular assist device (“LVAD”) to the aortic wall, have been quantified as a means to understanding the mechanism of aortic valve insufficiency. See, e.g., Callington et al. “Computational fluid dynamic study of hemodynamic effects on aortic root blood flow of systematically varied left ventricular assist device graft anastomosis design,” J. Thorac Cardiovasc Surg. 2015 September; 150(3):696-704. doi: 10.1016/j.jtcvs.2015.05.034. Epub 2015 May 15, the contents of which are incorporated herein by this reference.

Thus provided is the automatic adjustment of the impeller speed and pulsations of the pulsating cuff based upon real time pressure differentials and other data from the implanted sensors, which are placed in strategic positions. In a preferred embodiment, the sensors are placed above and below the catheter, cuffs, or stents. Such an embodiment optimizes flow by also timing pulsations of the pulsating cuff and impeller speed/angle with patient conditions and needs, including synchronization thereof with optimal real time pulsatile flow.

With various prior art devices, clinicians need to make manual adjustments of up to a dozen times an hour around the clock to be able to manage circulatory assist support based upon a chosen constant aortic pressure differential range or other sensing parameters. In contrast, the described system can be managed automatically and more frequently with the intention of improving patient outcomes. Furthermore, in designing a wireless power-based system and taking into consideration the risk of mechanical breakdown, demands on the system can be reduced (when patient conditions permit) for a time, allowing the device to “cool off” or “rest.” Inversely, the circulatory assist support can be turned up when demands dictate a genuine need and not before.

Such a system permits patient treatment to be customized on a real time personalized basis to provide superior outcomes for patients (e.g., those suffering from cardio-renal dysfunction in the advanced stages of heart failure).

In one embodiment of the system, a first impeller stent pump is positioned in the subject's ascending thoracic aorta, which unloads blood from the subject's heart (e.g., the first impeller stent pump is positioned to withdraw blood from the subject's left ventricle). In such an embodiment, a pulsating, partially ePTFE (expanded polytetrafluoroethylene) covered stent graft with three (3) pulsating bands is preferably positioned in the aorta downstream from the positioned first impeller stent pump. Also, a second impeller stent pump is positioned further downstream in the subject's descending aorta, just above the subject's renal arteries.

Such a three (3) band pulsating aortic stent graft typically a stent made of flexible compliant material (like an intra-aortic balloon pump (“IABP”) catheter balloon turned inside out). Two of the bands are always firmly against the aorta wall and only one band squeezes inward into the aorta at a time.

Left ventricular unloading is known and described, e.g., in Watanabe et al. “Left Ventricular Unloading Using an Impella CP Improves Coronary Flow and Infarct Zone Perfusion in Ischemic Heart Failure,” J Am Heart Assoc. 2018; 7:e006462. DOI: 10.1161/JAHA.117.006462, Esposito et al. “Left Ventricular Unloading Before Reperfusion Promotes Functional Recovery After Acute Myocardial Infarction” Journal of the American College of Cardiology, Vol. 72, issue 5, pp. 501-514 (Jul. 31, 2018), Saku et al. “Total Mechanical Unloading Minimizes Metabolic Demand of Left Ventricle and Dramatically Reduces Infarct Size in Myocardial Infarction,” doi.org/10.1371/journal.pone.0152911 (2016), Kapur et al. “Mechanically Unloading the Left Ventricle Before Coronary Reperfusion Reduces Left Ventricular Wall Stress and Myocardial Infarct Size,” Circulation. 128. 10.1161/CIRCULATIONAHA.112.000029. (June 2013), http://dx.doi.org/10.1161/CIRCULATIONAHA.112.000029, and “Acute Cardiac Unloading and Recovery,” Interventional Cardiology Review 2017; 12(2 Suppl. 2):1-28. See, also, Esposito M L, Kapur N K. “Acute mechanical circulatory support for cardiogenic shock: the ‘door to support’ time,” F1000Research. 2017; 6: 737. doi:10.12688/f1000research.11150.1.

The real time auto adjustment technology should serve patients, such as those that have physiologic hemodynamic changes due to things as simple as sleep and exercise with advanced heart failure changes in edema levels and modulation of the pump thrust, volume and impeller speed may serve these patients well. By enabling real time automatic adjustments of circulatory assist pump controls to adjust to the constant turbulent changes in hemodynamic and edema conditions that occur on an ongoing basis in, e.g., advanced heart failure patients.

A preferred aortic stent cage (FIG. 7) is designed to minimize hemolysis, while maximizing flow and stability. It is designed to avoid thick elements and to avoid razor cutting. It maximizes stability and positioning of the system. It presently serves as the best protection against the impeller blade(s) hitting the aortic wall.

The wire diameter of the stent cage circulatory assist catheter should be from about 0.015 to about 0.022 inches; preferably about 0.018 inches. Such a diameter is not too thin to cut blood cells and not too thick to ram them hard damaging them.

The catheter and drive shaft are designed to reduce risk of mechanical breakdown by having fewer bearings, which requires less fluid lubrication and flush. They are also designed to ease placement and minimize FR size. The drive shaft lubrication system preferably has minimal bearings and utilizes liquid cooling and an expanded polytetrafluoroethylene (“ePTFE”) liner. ePTFE is commercially available from, e.g., W. L. Gore & Associates.

Preferably, the impeller rotates at a number of revolutions, which is less than 10,000 rpm, preferably on the order of 4,500 rpm. Lower RPMs reduce the risk of mechanical failure and also reduce power needs. This can be important since, as reported by Kormos et al. “Left Ventricular Assist Device Malfunctions: It's More Than Just The Pump,” CIRCULATIONAHA.117.027360, originally published Jul. 3, 2017 (doi.org/10.1161/CIRCULATIONAHA.117.027360), 19% of patients suffered battery failure with the Heartmate II over 3 years. Heartmate II (Thoratec Corporation) is a heart pump called a left ventricular assist device (LVAD), which was designed to assist the left side of the heart to pump the blood a body needs. Furthermore, 21% of the HeartMate II patients were reported to have had driveline failure with the HeartMate II. The herein described preferred device having liquid cooled, minimal bearing system with ePTFE line and hydrophilic coated drive shaft act to reduce driveline failures.

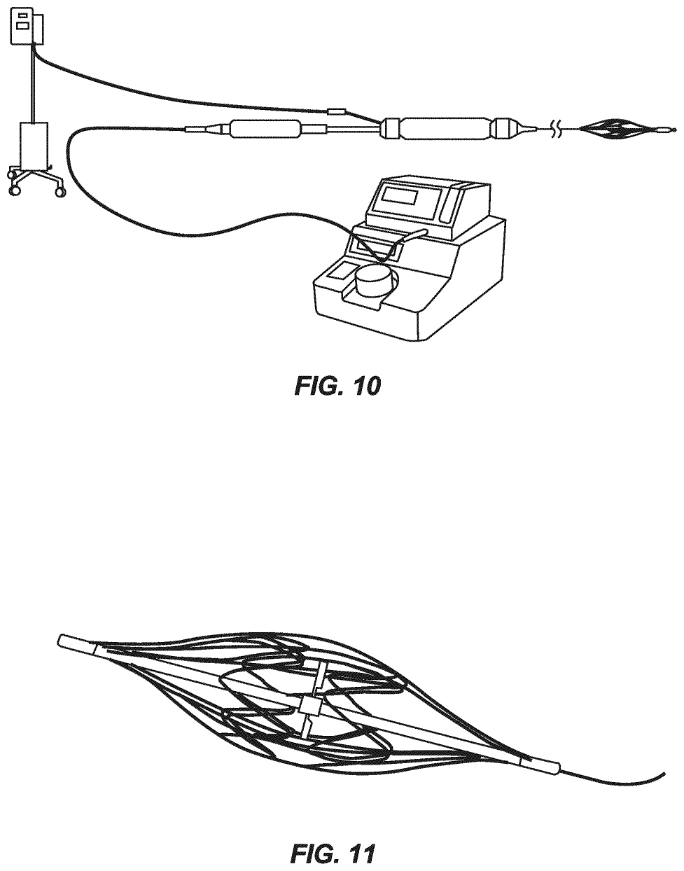

As depicted in FIG. 10, the system generally includes a motor and controller, a catheter (e.g., a Biomerics Advanced Catheter from Biomerics, Brooklyn Park, Minn.) that includes the catheter, catheter connector, drive shaft, handle, propeller/impeller, and tip, and a stent cage or frame, e.g., adapted through laser welding for application. As shown in FIG. 14, a “Biomerics Advanced Catheter” has a catheter handle, catheter connector, drive shaft, impeller encaged within the stent cage, and catheter tip.

A preferred handle (FIG. 14) typically has two wheels to manipulate the impeller and deployment of the stent. The first wheel may thus be used to remove the sheath and expose the (closed) impeller pump. The stent typically has a diameter of 20 mm, while the “opened” device typically has a diameter of 22 mm.

A preferred motor is not contained within the patient's circulation (FIG. 10). A preferred controller controls the speed and rpm of the device.

In FIG. 10, the propeller-driven “pump” includes a driveline (“sheath”) and the impeller. A proximal sheath is a driveline connecting the pump to a handle (or distal sheath/driveline). The distal driveline connects to a console motor (e.g., depicted is a light and quiet external BLDC motor that is mechanically and thermally isolated and uses a flexible interconnect for ease of positioning, a motor drive control unit and central alarm box). A console extension cable may be used to connect the console to the motor. The console thus may control operation of the pump. An infusion pump (the one depicted in the figure is an off the shelf IVAC/Infusion pump system using standard infusion tubing that terminates in a male Luer connector; medical grade UPS for transport and system power back up) may be used to control the volume of fluid entering the pump (above the distal sheath/driveline). The depicted distal and proximal sheath drivelines use Nitinol inner shafts, a positive action handle for accurate deployment, retraction, and locking of impeller blades. Infusion tubing is then used to deliver fluid as desired.

Such a system can generally involve two different embodiments. First, the temporary circulatory assist support pump(s) is/are placed on the tip of endovascular aortic catheter. Second, the system may include a removable chronic wireless powered implant circulatory assist pump within an aortic stent.

Such a system is designed to reduce heart work load and improve perfusion, improve renal function, normal the hemodynamics of acute decompensating heart failure patients, support heart regeneration procedures, help patients recover from cardiogenic shock, reduce risks associated with percutaneous catheterization interventions (“High Risk PCI”), help patients on the amputation list. Such a system is designed to reduce end diastolic pressure and to reduce end diastolic volume. It is further designed to reduce oxygen demand of myocardium.

Such a system utilizes a relatively straightforward aorta position insertion and is relatively stable over time. It promptly provides hemodynamic support. It is designed to minimize heart valve damage and to minimize coronary re-perfusion injury. It is designed to have low shear stress on blood, and minimize hemolysis.

The wireless power embodiment is designed to reduce infection risk compared to external drive line systems. Also, the wireless power option helps improve the patient's quality of life.

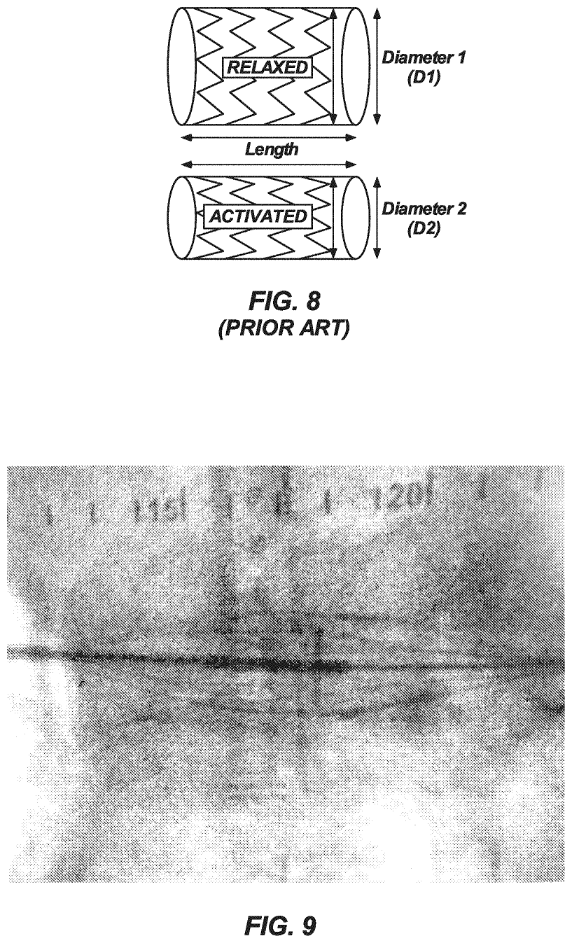

Preferably, the system is utilized with an upper aorta pulsating aortic cuff stent graft (FIG. 8), which improves the total flow of the system, improves hemodynamics, (via the pulsatile flow) improves the release of beneficial proteins for organ health, and reduces RPMs needed by the impeller to reach desired flow rates. A preferred system includes at least three (3) pulsating aortic cuffs on a flexible mesh aortic stent. Pulsating cuffs placed on the top, middle, and bottom of a flexible mesh stent may be controlled via an external abdominal belt.

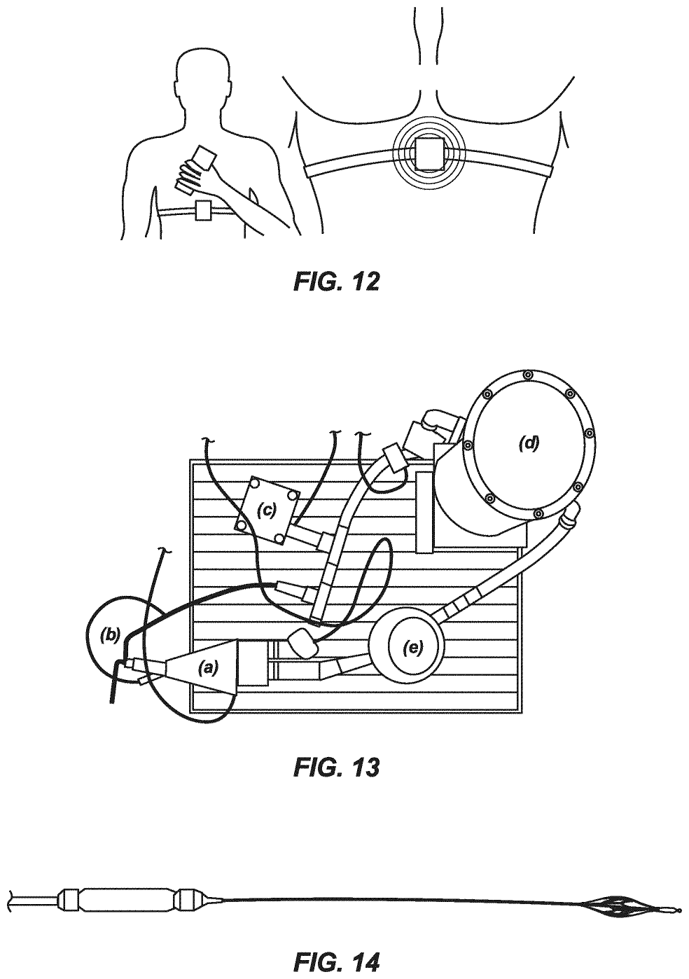

Pulsating electromagnetic waves may be, e.g., delivered non-invasively from an abdominal belt (e.g., FIG. 12) in direct communication with the aortic blood flow.

In certain embodiments, the wirelessly driven impeller is contained within a high aortic force protective cage stent (FIG. 7) is placed within such an upper aorta pulsating aortic cuff stent graft in the patient.

The system preferably combines the upper aorta pulsating aortic stent graft with a lower aorta impeller pump within a bare aortic stent to optimize flow with the least power and the least RPMs. Other pulsating aortic stent grafts are on the outside of the aorta, while the described is preferably on the inside. This is more effective, with less variability

FIG. 7 shows an embodiment of the device, where a wirelessly driven impeller is contained within a protective aortic cage stent. The depicted device has a cam lobe to release and retract the shaped impeller (e.g., 14.5 mm width) blades, two bearings, and an open protective aortic cage stent. The elements of the protective aortic cage stent are rounded. The depicted device utilizes relatively low RPM speed (7,500 vs. 10,000 to 33,000), maintains arterial pulsatility, and preferably uses the entire aorta of the patient (with the use of a protective aortic cage stent of, e.g., 23.5 mm).

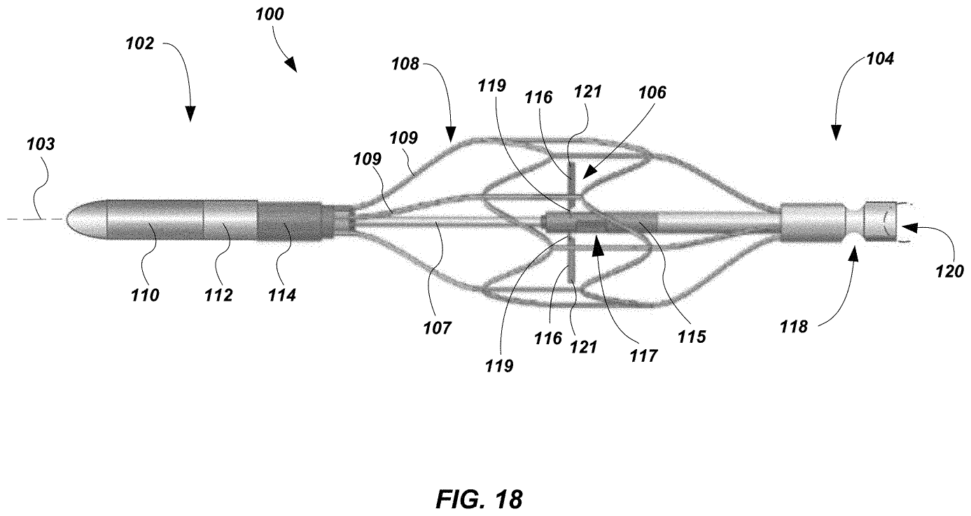

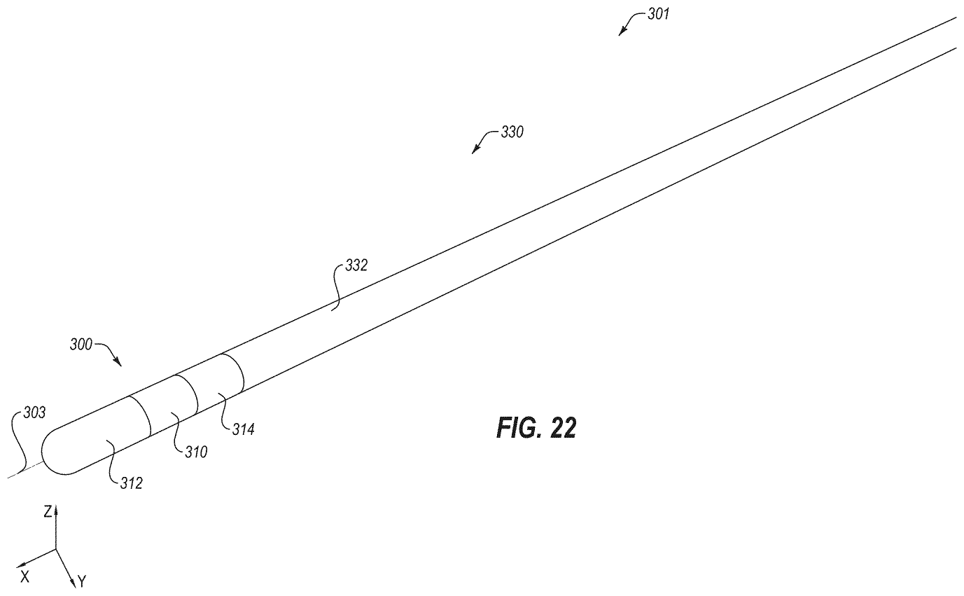

In some embodiments, such as shown in FIG. 18, a wireless circulatory assist pump 100 may be configured to be deployed into and removed from a vein or artery (e.g., the aorta) via one or more catheters (e.g., FIG. 22 below). The wireless circulatory assist pump 100 may comprise a distal end 102, a proximal end 104 (e.g., docking end), and an impeller 106 enclosed within a stent cage 108 therebetween. The wireless circulatory assist pump 100 may further comprise a battery 110, circuitry 112, and a motor 114. The circuitry 112 may comprise a wireless charging circuit, a communications circuit, and a control circuit. As shown in FIG. 18, the battery 110, the circuitry 112, and the motor 114, may all be located at or near the distal end 102, but it will be understood that one or more, or all, of the battery 110, the wireless charging circuit, the communications circuit, the control circuit, and the motor 114, may alternatively be located at or near the proximal end 104. The motor 114 may be connected to a driveline 107 that includes the impeller 106. The motor 114 may further be configured to rotate the driveline 107, including the impeller 106, within the stent cage 108 to facilitate blood circulation within a subject's blood vessel(s) (e.g., aorta).

The proximal end 104 may be configured to connect to connect to a catheter including an outer sheath configured to move axially relative to the wireless circulatory assist pump 100 to encompass and constrain the stent cage 108 and the impeller blades 116 therein during insertion and removal of the wireless circulatory assist pump 100 from a subject's blood vessel(s) (e.g., aorta). To connect to the catheter for insertion and removal, the proximal end 104 of wireless the circulatory assist pump 100 may include a circumferential groove 118 configured to receive a corresponding portion of the insertion and removal catheter. The proximal end 104 of the wireless circulatory assist pump 100 may additionally include a hemispherical groove 120 configured to receive a corresponding portion of the insertion and removal catheter.

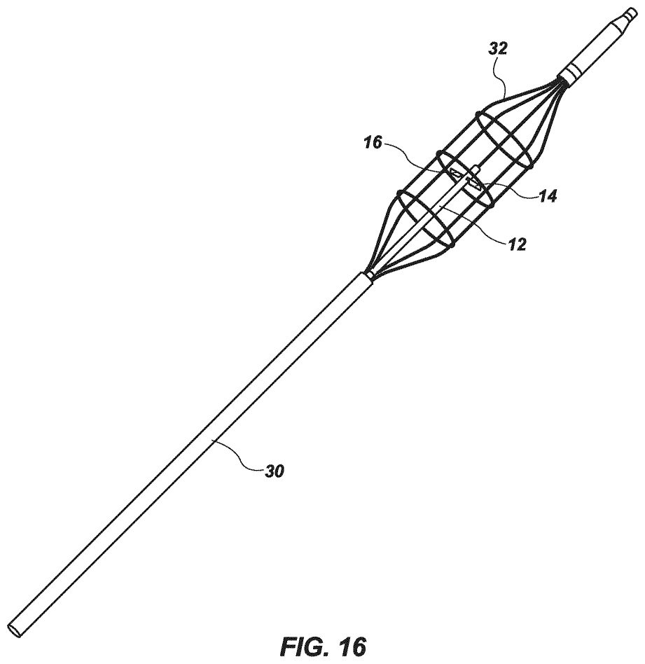

In some embodiments, the stent cage 108 may be similar to the stent cage 32 described previously herein with reference to FIGS. 16 and 17. Accordingly, the stent cage 108 may be configured to expand to securely position the wireless circulatory assist pump 100 in a patient's aorta, while maintaining the pulsatility of the aorta. Additionally, the stent cage 108 may be collapsed and stowed for placement and removal of the wireless circulatory assist pump 100.

In some embodiments, the stent cage 108 may be made of one or more materials configured to selectively change shape. For example, the stent cage 108 of the wireless circulatory assist pump 100 may be configured to selectively change to a pre-determined expanded (e.g., deployed) state. The stent cage 108 may comprise one or more temperature sensitive shape memory materials configured to change to a predetermined expanded (e.g., deployed) state in response to the shape memory material being at about and/or above a transition temperature. As non-limiting examples, shape memory materials may include shape memory alloys, such as Nitinol, and shape memory polymers such as polyether, polyacrylate, polyamide, polysiloxane, polyurethane, polyethylene, methyl-methacrylate (MMA), polyethylene glycol (PEG), polyethylene glycol dimethacrylate (PEGDMA), polyether amide, polyether ester, or urethane-butadiene copolymer. In addition, the stent cage 108 may include a hydrophilic coating such as those available from Coloplast Manufacturing, US LLC located at 1940 Commerce Drive, North Mankato, Minn. 56002.

The transition temperature for shape memory alloys is the austenite transition temperature (T_{A}), and the transition temperature for shape memory polymers may be either the high glass transition temperature (T_{G}) or the intermediate melting temperature (T_{M}). Temporary stresses and/or strain within the shape memory material may be removed in response to the shape memory material being at and above the transition temperature. In addition, superelasticity (e.g., for shape memory alloys) and visco-elasticity (e.g., for shape memory polymers) of shape memory material may facilitate constructions of circulatory assist devices and systems with fewer moving parts. For example, the stent cage 108 may be configured to expand without the use of a mechanical mechanism (e.g., cam, spring, etc.) that may otherwise be used to reliably expand the stent cage 108.

The stent cage 108 may be made of a shape memory material tailored to have a transition temperature (e.g., T_{A} for shape memory alloys, or either To or T_{M} for shape memory polymers) to be about the internal body temperature of the subject (e.g., a mammal, such as a human). For example, the transition temperature of the shape memory material may be from about 35° C. to about 40° C. (e.g., about 37° C.). As described in U.S. Pat. No. 4,283,233 to Goldstein et al. (Aug. 11, 1981), the contents of which is incorporated herein by this reference, shape memory alloys, such as Nitinol, can be engineered to include an austenite transition temperature just below the normal human body temperature, thus allowing for the shape memory effect and super elasticity effects to occur in conjunction with the circulatory assist devices and systems of the present disclosure due to body heat. Similarly, U.S. Patent Pub. No. 2009/0248141 to Shandas et al. (Published Oct. 1, 2009), incorporated herein by this reference, describes a method of tailoring the transition temperature of shape memory polymers to allow recovery at, above, or below the human body temperature of 37° C.

The stent cage 108 is expandable and collapsible to provide a narrow profile for introduction into the subject's blood vessel(s) (e.g., aorta) to a desired position (e.g., just above the subject's renal arteries in the descending aorta).

In embodiments in which the stent cage 108 comprises a temperature sensitive shape memory material, the stent cage 108 may be trained to “remember” a predetermined state, such as the expanded state (FIG. 18) when the temperature of the stent cage 108 are at or above the transition temperature of the respective shape memory material. Accordingly, after inserting the wireless circulatory assist pump 100 within a patient, stent cage 108 may rise to a temperature at or above the transition temperature of the respective shape memory material, and the stent cage 108 may transition to the predetermined state. The stent cage 108 may reach the transition temperature while the circulatory assist pump 100 is still in a collapsed state within a sheath of a catheter and conform to the shape thereof. In such instances, the stent cage 108 may be biased toward the predetermined state such that the stent cage 108 springs into the predetermined state (e.g., the expanded state) when the sheath of the catheter is withdrawn.

The stent cage 108 may be of a size and shape to allow a highly open blood flow when the stent cage 108 is in an expended state within the subject's blood vessel(s) (e.g., aorta). Furthermore, the stent cage 108 may be configured to provide a radial force against an inner wall of the subject's aorta when the stent cage 108 is in the expanded state such that the stent cage 108 is positionally secured to the inner wall of the subject's aorta and such that the stent cage 108 flexes with a natural pulsatility of the subject's aorta.

The stent cage 108 comprises wire-like elements 109 that extend from the distal end 102 and the proximal end 104 toward a center of the wireless circulatory assist pump 100. The stent cage 108 further comprises reinforcing wire-like elements 109 arranged circumferentially in a sinusoidal wave pattern. For example, wire-like elements 109 extending from the distal end 102 are connected to a reinforcing wire-like element 309 proximate the proximal end 304, and the wire-like elements 109 extending from the proximal end 104 are connected to a reinforcing wire-like element 109 proximate the distal end 102. Axial wire-like elements 109 overlap with circumferential reinforcing wire-like elements 109 to provide a sufficiently rigid structure to secure to the inner wall of the subject's blood vessel(s) (e.g., aorta), while also maintaining flexibility. Accordingly, the stent cage 108 may exhibit a balance of flexibility and rigidity such that the stent cage 108 is configured to maintain an axial position within the subject's aorta and radially flex with the natural pulsatility of the subject's aorta.

The wire-like elements 109 of the stent cage 108 may exhibit any desired size and shape. In some embodiments, the wire-like elements 109 exhibit a rounded shape. For example, a cross-section the wire-like elements 109 may exhibit the shape of an oval or circle. Additionally, the wire-like elements 109 may exhibit a size (e.g., diameter) within a range of from about 0.1 millimeters (mm) to about 1 mm, such as within a range of from about 0.2 mm to about 0.7 mm, within a range of from about 0.3 mm to about 0.6 mm, (e.g., about 0.5 mm).

When the stent cage 108 is in the expanded state within the subject's aorta, the stent cage 108 is configured to provide a radial force against the inner wall of the subject's aorta. For example, the stent cage 108 may be made of a shape memory material with a “remembered” shape being the stent cage 108 in the expanded state. Accordingly, the stent cage 108 may be biased radially outward after being inserted into the subject's aorta and being at or above the transition temperature (e.g., about 37° C.). When the stent cage 108 is in the expanded state within the subject's aorta, the radial force provided by the wire-like elements 109 of the stent cage 108 against the inner wall of the subject's aorta may be within a range of from about 0.1 Newtons (N) to about 1 N, such as within a range of from about 0.2 N to about 0.8 N, within a range of from about 0.3 N to about 0.7 N, within a range of from about 0.4 N to about 0.6 N (e.g., about 0.5 N). The radial force applied by the wire-like elements 109 of the stent cage 108 to the inner wall of the subject's aorta may embed the wire-like elements 109 within the inner wall of the subject's aorta. In some embodiments, the radial force applied by the wire-like elements 109 of the stent cage 108 (in the expanded state) to the inner wall of the subject's aorta is configured to embed an entire thickness of the wire-like elements 109 into the inner wall of the subject's aorta such that the opening defined within the embedded wire-like elements 109 of the stent cage 108 is substantially the same size (e.g., area) as the subject's aorta. The wire-like elements 109 of the stent cage 108 being flush within subject's aortic wall may lessen the risks associated with turbulence and obstruction to blood flow.

Accordingly, the stent cage 108 may exhibit a balance of flexibility and rigidity such that the stent cage 108 is configured to maintain an axial position within the subject's aorta and radially flex with the natural pulsatility of the subject's aorta. In other words, when the stent cage 108 is in the expanded state within the subject's aorta, the stent cage 108 is designed to inhibit (e.g., prevent) movement along the subject's aorta, meanwhile enabling radial flexing to match the natural pulsations of the subject's aorta. The stent cage 108 being rigid (e.g., sturdy) enough to keep the position of the wireless circulatory assist pump 100 positionally stable, while also being flexible enough to flex with aortic wall movements of the subject may provide certain advantages compared to conventional devices. For example, the stent cage 108 may facilitate proper protein expressions for kidney health and modulating hypertension. Furthermore, the stent cage 108 is capable of being wirelessly powered in a reliable manner because the stent cage 108 will not slip out of place once the stent cage 108 is expanded and the wire-like elements 109 are embedded within the subject's aortic wall. In additional embodiments, the stent cage 108, in the expanded state, may distend the subject's aorta by an amount within a range from up to 2 mm (e.g., about 0.5 mm, about 1.0 mm, about 1.5 mm, or about 2 mm) to further secure the position of the stent cage 108 within the subject's aorta.

The motor 114 may be a miniature brushless direct current (DC) motor. For example, the motor 114 may be a miniature brushless DC motor such as available under the tradename “EC6” from Maxon Precision Motors, Inc. of Foster City, Calif. USA.

The battery 110 may be a rechargeable battery, such as a lithium-ion battery. For example, the battery 110 may be a 3 milliamp hour (mAh) lithium-ion battery available under the tradename “CONTIGO” from EaglePicher Technologies of Joplin, Mo. USA. For another example, the battery 110 may be a 3 mAh lithium-ion battery available under the tradename “MICRO3-QL0003B” from Quallion LLC of Sylmar, Calif. USA. It will be understood, however, that the battery 110 may be of any suitable chemistry and/or type, including non-chemical electric power storage devices, such as a capacitor (e.g., a supercapacitor, ultracapacitor, or double-layer capacitor).

The wireless charging circuit may produce an electric current in response to an applied electric field, magnetic field, and/or electromagnetic field, which may be utilized to charge the battery 110. For example, the wireless charging circuit may comprise an induction coil and energy may be transferred to the wireless charging circuit via inductive coupling. For another example, the wireless charging circuit may comprise one or more antennas and energy may be transferred to the wireless charging circuit via electromagnetic waves (e.g., radio waves).

The communication circuit may be configured to send and receive data via wireless communication. For example, the communication circuit may be configured to send and receive data utilizing radio communication (e.g., WiFi, Bluetooth, etc.). In some embodiments, the communication circuit may be utilized to send data collected from one or more sensors of the wireless circulatory assist pump 100. For example, the communication circuit may be utilized to send data relating to the rotational speed of the pump, upstream and downstream fluid pressures, battery charge status, motor status, impeller status, and/or other measured conditions.

The control circuit may be utilized to control certain operations of the wireless circulatory assist pump 100. In some embodiments, the control circuit may be utilized to control the rotational speed of the motor 114, the shape of the impeller 106, the deployment of the impeller blades 116, the stowing of the impeller blades 116, the angle of the impeller blades 116, and/or other operations of the circulatory assist pump 100.

In some embodiments, the circulatory assist pump 100 may comprise one or more application-specific integrated circuit (“ASIC”) chips. For example, one or more of the charging circuit, the communication circuit, and the control circuit may be provided as one or more ASIC chips.

The impeller 106 includes a casing 115 and the impeller blades 116 configured to move relative to the casing 115 from a deployed state (e.g., expanded or open state), as shown, to a stowed state (e.g., collapsed or closed state) (see, e.g., FIGS. 2 and 22), and vice versa. The impeller blades 116 may additionally be configured to move to states between the deployed state and the stowed state (e.g., to a partially deployed state, also referred to as a partially expanded state or partially open state). The casing 115 may be secured to (e.g., fastened to or integral with) the driveline 107 such that one revolution of the driveline 107 corresponds to one revolution of the impeller 106 (e.g., the casing 115 and the impeller blades 116).

As shown in FIG. 18, the impeller blades 116 include fixed ends 119 secured to (e.g., fastened to or integral with) the casing 115, and free ends 121 opposite the fixed ends 119. For example, the impeller blades 116 may be integral with a narrow section of the driveline 107 that extends through a central portion of the casing 115, and ends of the casing 115 may be secured to larger sections of the driveline 107 on either end of the casing 115.

In the deployed state, as shown, the free ends 121 and remainder of the impeller blades 116 are oriented substantially perpendicular to the casing 115, the driveline 107, and a central longitudinal axis 103 of the driveline 107. In the stowed state, the free ends 121 and the remainder of the impeller blades 116 are received within pockets 117 of the casing 115 of the impeller 106 such that the impeller blades 116 are substantially parallel to the casing 115, the driveline 107, and the central longitudinal axis 103 of the driveline 107. The pockets 117 defined by the casing 115 may correspond to shapes of the impeller blades 116. Thus, when the impeller blades 116 are received within the pockets 117 of the casing 115, the outer surfaces of the impeller blades 116 may form a continuous surface with the casing 115. In the partially deployed state, the free ends 121 and the remainder of the impeller blades 116 are oriented somewhere between being substantially perpendicular to and substantially parallel to the casing 115, the driveline 107, and the central longitudinal axis 103 of the driveline 107.

As shown in FIG. 18, the free ends 121 of the impeller blades 116 of the impeller 106 may be configured to rotate outward from the pockets 117 of the casing 115 and toward the distal end 102 of the wireless circulatory assist pump 100. Conversely, the free ends 121 of the impeller blades 116 of the one or more impellers 106 may be configured to rotate and toward the proximal end 104 to stow the impeller blades 116 in the pockets 117 of the casing 115. In additional embodiments, the free ends 121 of the impeller blades 116 of the impeller 106 may be configured to rotate outward from the pockets 117 of the casing 115 and toward the proximal end 104 of the wireless circulatory assist pump 100.

In some embodiments, the impeller 106 may transition between the deployed state, the partially deployed state, and the stowed state by axially translating the impeller 106 (e.g., the casing 115 and the impeller blades 116) relative to the driveline 107. For example, the driveline 107 may include an internal rod portion at the distal end of the casing 115 that interacts with cam portions at the fixed ends 119 of the impeller blades 116 to open the impeller blades 116. Axial translation of the casing 115 (e.g., toward the distal end 102) relative to the driveline 107 may open the impeller blades 116, and axial translation of the casing 115 (e.g., toward the proximal end 104) in the opposite direction relative to the driveline 107 may stow the impeller blades 116.

In additional embodiments, the impeller 106 (e.g., the impeller blades 116) may comprise shape changing materials such that the impeller 106 (e.g., the impeller blades 116) is configured to change shapes in one or more various ways. For example, the impeller 106 (e.g., the impeller blades 116) may be configured to selectively move between the deployed state, the deployed state, and the partially deployed state. In some embodiments, the impeller 106 (e.g., the impeller blades 116) may be configured to rotate or twist to selectively vary the pitch of the impeller blades 116. In additional embodiments, the impeller blades 116 may be configured to bend to selectively alter the curvature of the impeller blades 116. Accordingly, the impeller 106 (e.g., the casing 115 and the impeller blades 116) may substantially free of any fasteners or separate mechanical mechanisms (e.g., cam mechanisms, springs, etc.). Furthermore, the impeller blades 116 being capable of opening to a deployed state or partially deployed state may enable the use of the same impeller blades 116 in different sized blood vessel(s) within the subject. For example, each of the impeller blades 116, may exhibit a length (defined by a distance along the impeller blade 116 from the fixed end 119 to the free end 121) within a range of from about 5 mm to about 15 mm, such as about 5 mm, about 6 mm, about 7 mm, about 8 mm, about 9 mm, about 10 mm, about 11 mm, about 12 mm, about 13 mm, about 14, mm or about 15 mm. Thus, if the subject's blood vessel (e.g., aorta) has a diameter of about 22 mm, any of the aforementioned sizes of impeller blades 116 may be used because the impeller blades 116 can operate in a partially deployed state (e.g., not substantially perpendicular to the central longitudinal axis 103 of the impeller 106, the driveline 107, and/or the casing 115).

Certain impeller shapes and curvatures can optimize blood flow and minimize hemolysis in both chronic implantable and temporary circulatory assist devices. Most of these ideal optimized shapes, however, are not practical for delivery via a percutaneous non-surgical delivery catheter. Additionally, not one impeller shape appears to be ideal for all circumstances to best meet patient needs at all times. Accordingly, impellers 106 according to embodiments of the disclosure may change shape on demand to meet patient needs as they arise that can be delivered and removed without surgery. Traditionally, these ideal impeller shapes are fixed in shape and cannot be changed without mechanically making a change in manufacturing.

In some embodiments, the impeller 106 may be configured for shape changing on demand for use in circulatory assist pumps 100 that substantially eliminate or reduce disadvantages of devices with fixed shape impellers. In particular, the impeller shape may be changed to lengthen its diameter to increase flow and can be twisted into an optimized shape to improve flow, minimize turbulence, minimize thrombosis risk, and minimize hemolysis and can be returned to original shape also on demand to facilitate removal via percutaneous catheter means, not surgery.

In some embodiments, the impeller blade length, shape, twist, curvature and/or overall form may be changed on demand utilizing an electrical field to result in bending deformation to a pre-determined different shape and size, and can be returned to the original shape also upon demand to facilitate removal percutaneously if desired.

In further embodiments, the impeller blade length, shape, twist, curvature and/or overall form may be changed on demand utilizing a temperature sensitive shape memory material to result in bending deformation to a pre-determined different shape and size, and can be returned to original shape also upon demand such as, for example, by flushing temporarily with a cold solution to facilitate removal percutaneously if desired. The impeller 106, including the casing 115 and/or the impeller blades 116 may be made of a shape memory alloy, such as Nitinol, and/or a shape memory polymer such as polyether, polyacrylate, polyamide, polysiloxane, polyurethane, polyethylene, methyl-methacrylate (MMA), polyethylene glycol (PEG), polyethylene glycol dimethacrylate (PEGDMA), polyether amide, polyether ester, or urethane-butadiene copolymer. In a similar manner to the stent cage 108, as previously indicated, the impeller 106 (e.g., the casing 115 and/or the impeller blades 116) may be made of a shape memory material tailored to have a transition temperature (e.g., T_{A} for shape memory alloys, or either To or T_{M} for shape memory polymers) to be about the internal body temperature of the subject (e.g., a mammal, such as a human). For example, the transition temperature of the shape memory material may tailored to be from about 35° C. to about 40° C. (e.g., about 37° C.).

In embodiments in which the impeller 106 (e.g., the casing 115 and/or the impeller blades 116) comprise a temperature sensitive shape memory material, the impeller 106 (e.g., the impeller blades 116) may be trained to “remember” a predetermined state, such as the deployed state (FIG. 18) or a partially deployed state when the temperature of the impeller 106 (e.g., the impeller blades 116 and, optionally, the casing 115) is at or above the transition temperature of the respective shape memory material. Accordingly, after inserting the wireless circulatory assist pump 100 within a patient, the impeller 106 may rise to a temperature at or above the transition temperature of the respective shape memory material, and the impeller 106 may transition to the predetermined state. In a similar manner, the impeller blades 116 may be trained to “remember” a predetermined blade length, shape, twist, curvature and/or overall form. The impeller 106 (e.g., the casing 115 and/or the impeller blades 116) may reach the transition temperature while the circulatory assist pump 100 is still in a collapsed state within a sheath of a catheter and conform to the shape thereof. In such instances, the impeller 106 (e.g., the impeller blades 116) may be biased toward the predetermined state such that the impeller 106 (e.g., the impeller blades 116) springs into the predetermined state when the sheath of the catheter is withdrawn and the stent cage 108 is expanded.

In yet further embodiments, the impeller blade length, shape, twist, curvature and/or overall form may be changed on demand utilizing both an electrical field to result in bending deformation to a pre-determined different shape and size, and a temperature sensitive shape memory alloy, such as nitinol, to result in bending deformation to a pre-determined different shape and size, and can be returned to original shape also upon demand, such as, for example, by flushing temporarily with a cold solution.

In additional embodiments, the impeller blades 116 may comprise a frame comprised of a temperature sensitive shape memory alloy connected to one or more sheets of electro active polymer. For example, the impeller blades 116 may have a structure similar to airfoils used in the construction of airplanes in early aviation. Accordingly, the length, shape, twist, curvature and/or overall form may be changed on demand utilizing the frame comprised of a temperature sensitive shape memory alloy and the one or more sheets of electro active polymer.

In additional embodiments, the impeller 106 may comprise a shape change impeller 106A (e.g., an adjustable or extendible impeller) having impeller blades 116A that may comprise a frame 200 comprised of a resilient material connected to one or more sheets of electroactive material 202, such as an electroactive polymer or similar material. For example, the impeller blades 116A may have a structure similar to that of a dragonfly wing. The frame 200 of the impeller blades 116A may include corrugated regions 204, which may have a spring-like (e.g., helical) or pleated configuration. In an initial state, as shown in FIG. 20A, the corrugations or coils of the corrugated regions 204 of the impeller blades 116A may be relatively closely spaced. For example, the impeller blades 116A may have an overall length of about 12.5 mm in the initial state. In certain embodiments, in response to applied energy (e.g., an applied electrical field), the electroactive material 202 may expand in length. The corrugated regions 204 of the frame 200 may accommodate the change in shape of the electroactive material 202 by flattening the corrugations or spacing the helical coils, as shown in FIG. 20B; causing the overall length of the impeller blades 116A to increase. For example, the impeller blades 116A may increase to a length of about 15.5 mm or longer in some embodiments. Accordingly, the length, shape, twist, curvature and/or overall form may be changed on demand utilizing the frame comprised of a temperature sensitive shape memory alloy.

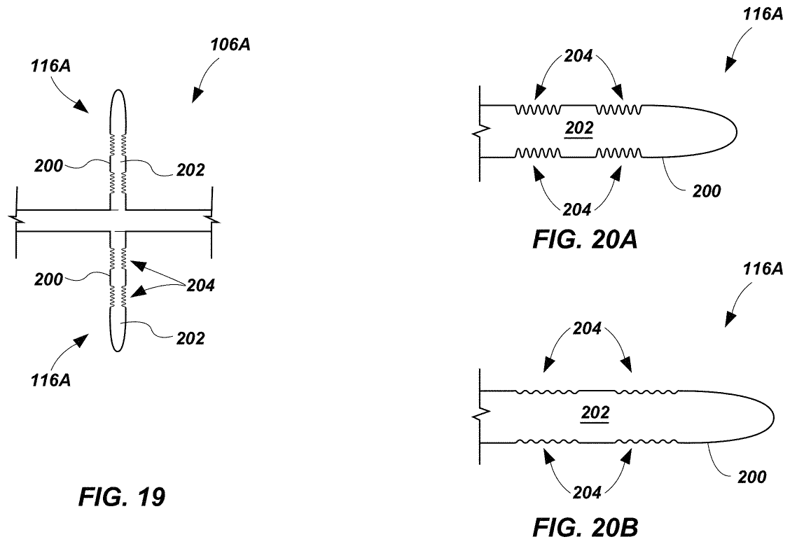

In yet additional embodiments, such as shown in FIG. 19, the impeller 106 may comprise a shape change impeller 106A having impeller blades 116A that may comprise a frame 200 comprised of a temperature sensitive shape memory alloy connected to one or more sheets of electroactive material 202, such as poly-paraphenylene terephthalamide (KEVLAR® or TWARON®) or similar material. The frame 200 of the impeller blades 116A may include corrugated regions 204, which may have a spring-like (e.g., a helical shape) or pleated configuration. In an initial state, as shown in FIG. 20A, the corrugations of the corrugated regions 204 of the impeller blades 116A may be relatively closely spaced. For example, the impeller blades 116A may have an overall length of about 12.5 mm in the initial state. In certain embodiments, in response to applied energy (e.g., heat within the patient's body), the shape memory material of the corrugated regions 204 of the frame 200 activate, flattening the corrugations or stretching a helical coil, as shown in FIG. 20B; causing the overall length of the impeller blades 116A to increase. For example, the impeller blades 116A may increase to a length of about 15.5 mm or longer in some embodiments. Accordingly, the length, shape, twist, curvature and/or overall form may be changed on demand utilizing the frame comprised of a temperature sensitive shape memory alloy.

In some embodiments the shape change impeller may include both a shape memory alloy frame 200 with sheets of electroactive material 202, which may work in cooperation to provide a change in shape to the impeller blades 116A. Additionally, in some embodiments, the frame 200 may comprise a spring-like structure (not shown).

As the impeller blades 116A may be provided with a flexible and resilient frame 200 and electroactive material 202, the frame may be deformed in response to an applied force and may resiliently return to a predetermined shape. Accordingly, the impeller blades 116A may be drawn into and stowed within a sheath of a catheter and conform to the shape thereof in response to the force applied by the sheath as it is drawn over the impeller blades 116A. The impeller blades 116A may then resiliently return to the predetermined shape upon exiting the sheath of the catheter. Accordingly, the impeller blades 116A may be deformable between a stowed position (e.g., within a sheath of a catheter) and resilient to a deployed position (e.g., when installed within a patient) without the need of mechanical joints.

In further embodiments, a computational fluid dynamics simulation device may be utilized to analyze all available patient and device data and determine the ideal impeller shape, length, speed, angle of deflection, curvature, power usage, and more for the situation and goals at hand. Then, the impeller blade length, shape, twist, curvature, and/or overall form may be changed on demand or as needed utilizing an electrical field to result in bending deformation to a pre-determined different shape and size that is most ideal for a given situation.

In some embodiments, the impeller blade length, shape, twist, curvature, and/or overall form may be changed on demand utilizing a light activated shape change material to result in bending deformation to a pre-determined different shape and size in response to an applied light, and can be returned to the original shape also upon demand to facilitate removal percutaneously if desired.

As previously discussed, the distal end 102 of the circulatory assist pump 100 may house the battery 110, the wireless charging circuit, the communications circuit, the control circuit, and the motor 114. The end of the distal end 102 may have a smooth, generally dome shaped, leading end. This may prevent harm to the patient should the distal end 102 come into contact with the arterial wall, such as during an insertion or removal procedure. The distal end 102 may comprise a canister covering and sealing the components therein. In some embodiments, a titanium canister may cover and seal the distal end 102. In yet additional embodiments, the canister may comprise at least a portion that is made of a material that is transparent to certain frequencies of electromagnetic radiation, magnetic fields, and/or electrical fields, such as a ceramic or a polymer, to facilitate electromagnetic, electric, and/or magnetic communication between devices located outside of the patient's body and components within the distal end 102.

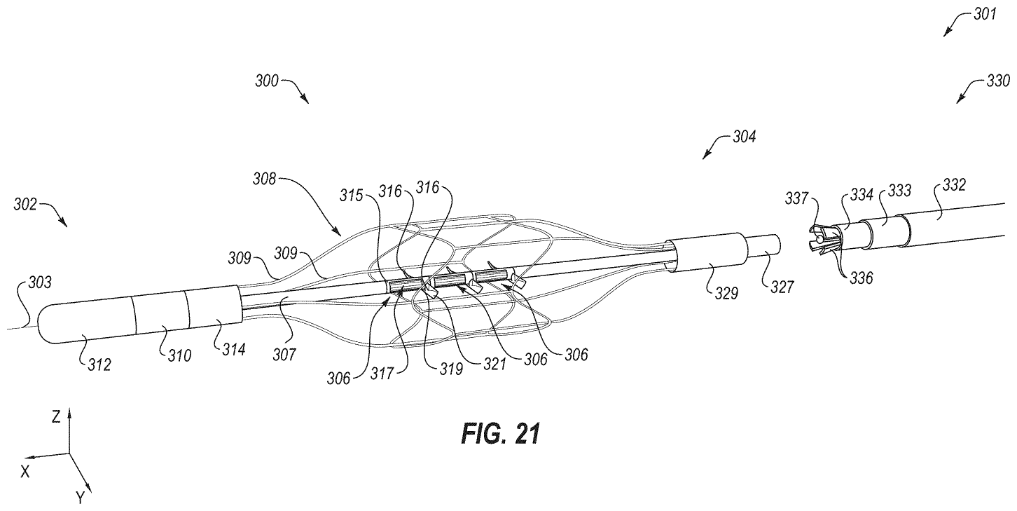

FIGS. 21 and 22 depict a circulatory assist system 301 that includes a wireless circulatory assist pump 300 and an insertion and removal catheter 330, in accordance with embodiments of the present disclosure. In FIGS. 21 and 22, and the associated description, functionally similar features (e.g., structures, materials) to those of the wireless circulatory assist pump 100 of FIG. 18 are referred to with similar reference numerals incremented by 100. To avoid repetition, not all features shown in FIGS. 21 and 22 are described in detail herein. Rather, unless described otherwise below, a feature as shown in FIGS. 21 and 22 designated by a reference numeral that is a 200 increment of the reference numeral of FIG. 18 will be understood to be substantially similar to the previously described feature of FIG. 18.

Referring collectively to FIGS. 21 and 22, the insertion and removal catheter 330 is configured to connect to the wireless circulatory assist pump 300 to facilitate introduction and removal of the wireless circulatory assist pump 300 into a subject's blood vessel(s) (e.g., aorta). The wireless circulatory assist pump 300 is configured to transition from a collapsed state (e.g., stowed state) (FIG. 22) to an expanded state (e.g., deployed state) (FIG. 21), and vice versa. To facilitate introduction and removal, the circulatory assist pump 300 is in the collapsed state (FIG. 22). After inserting the wireless circulatory assist pump 300 into the subject (e.g., within the subject's femoral artery) and positioning the wireless circulatory assist pump 300 in a desired location (e.g., above the subject's renal arteries in the descending aorta), the circulatory assist pump 300 may transition from the collapsed state (FIG. 22) to the expanded state (FIG. 21). After positioning the wireless circulatory assist pump 300 in the expanded state (FIG. 21), the insertion and removal catheter 330 may be disconnected from the wireless circulatory assist pump 300 and withdrawn from the subject. Furthermore, the wireless circulatory assist pump 300 may be activated (e.g., via wireless energy or battery power) to rotate impeller blades 316 of impellers 306 to facilitate blood circulation within the subject.

Referring now to FIG. 21, the wireless circulatory assist pump 300 generally includes a distal end 302, a proximal end 304 (e.g., docking end), and impellers 306 (three shown in FIG. 21) enclosed within a stent cage 308 between the distal end 302 and the proximal end 304. The wireless circulatory assist pump 300 may further include a battery 310, circuitry 312 and a motor 314. The circuitry 312 may comprise a wireless charging circuit, a communications circuit, and a control circuit. As shown in FIG. 21, the battery 310, the circuitry 312, and the motor 314, may all be located at or near the distal end 302, but it will be understood that one or more, or all, of the battery 310, the wireless charging circuit, the communications circuit, the control circuit, and the motor 314, may alternatively be located at or near the proximal end 304.

The end portion of the distal end 102 may have a smooth, generally dome shaped, leading end. This may prevent harm to the patient should the distal end 102 come into contact with the walls of the subject's blood vessel(s), such as during an insertion or removal procedure. The distal end 102 may comprise a canister (e.g., a hermetically sealed canister) covering and sealing the components therein. The circuitry 312 is at the distalmost part of the distal end 302 and enclosed within a hermetically sealed canister, the motor 314 is proximate the stent cage 308, and the battery 310 is interposed between the circuitry 312 and the motor 314. In some embodiments, a titanium canister may cover and seal the distal end 302. In yet additional embodiments, the canister may comprise at least a portion that is made of a material that is transparent to certain frequencies of electromagnetic radiation, magnetic fields, and/or electrical fields, such as a ceramic or a polymer, to facilitate electromagnetic, electric, and/or magnetic communication between devices located outside of the patient's body and components within the distal end 102.

The motor 314 may be connected to a driveline 307 that includes the impellers 306. The motor 314 may further be configured to rotate the driveline 307, including the impellers 306, within the stent cage 308 to facilitate blood circulation within a subject's blood vessel(s) (e.g., aorta).

The stent cage 308 may be substantially similar to the stent cage 108 described previously herein with reference to FIG. 18. Accordingly, the stent cage 308 may exhibit a balance of flexibility and rigidity such that the stent cage 308 is configured to maintain an axial position within the subject's aorta and radially flex with the natural pulsatility of the subject's aorta. In other words, when the stent cage 308 is in the expanded state within the subject's aorta, the stent cage 308 is designed to inhibit (e.g., prevent) movement along the subject's aorta, meanwhile enabling radial flexing to match the natural pulsations of the subject's aorta. The stent cage 308 being rigid (e.g., sturdy) enough to keep the position of the wireless circulatory assist pump 300 positionally stable, while also being flexible enough to flex with aortic wall movements of the subject may provide certain advantages compared to conventional devices. For example, the stent cage 308 may facilitate proper protein expressions for kidney health and modulating hypertension. Furthermore, the wireless circulatory assist device (pump) 300 is capable of being wirelessly powered in a reliable manner because the stent cage 308 will not slip out of place once the stent cage 308 is expanded and the wire-like elements 309 are embedded within and, optionally, distend the subject's aortic wall.

In some embodiments, the wireless circulatory assist pump 300 includes a single impeller 306. In additional embodiments, the wireless circulatory assist pump 300 includes multiple impellers 306 (e.g., two or more impellers) arranged in series on a common driveline 307. Each impeller 306 includes a casing 315 and impeller blades 316 configured to move relative to the casing 315 from a deployed state (e.g., expanded or open state), as shown, to a stowed state (e.g., collapsed or closed state) (see, e.g., FIGS. 2 and 22), and vice versa. The impeller blades 316 may additionally be configured to move to states between the deployed state and the stowed state (e.g., to a partially deployed state, also referred to as a partially expanded state or partially open state). The casings 315 may be secured to (e.g., fastened to or integral with) the driveline 307 such that one revolution of the driveline 307 corresponds to one revolution of the impellers 306 (e.g., the casings 315 and the impeller blades 316).

As shown in FIG. 21, the impeller blades 316 include fixed ends 319 secured to (e.g., fastened to or integral with) the casings 315 and/or the driveline 307, and free ends 321 opposite the fixed ends 319. For example, the impeller blades 116 may be integral with a narrow section of the driveline 107 that extends through a central portion of the casings 115, and ends of the casings 115 closest to the distal end 302 and the proximal end 304 may be secured to larger sections of the driveline 107.

In the deployed state, as shown, the free ends 321 of the impeller blades 316 are oriented substantially perpendicular to the casings 315, the driveline 307, and a central longitudinal axis 303 of the driveline 307. In the stowed state, the free ends 321 of the impeller blades 316 may be received within pockets 317 of the casings 315 of the respective impellers 306 such that the impeller blades 316 are substantially parallel to the casings 315, the driveline 307, and the central longitudinal axis 303 of the driveline 307. The pockets 317 defined by the casings 315 may correspond to shapes of the impeller blades 316. Thus, when the impeller blades 316 are received within the pockets 317 of the casings 315, the outer surfaces of the impeller blades 316 may form a continuous surface with the casings 315. In the partially deployed state, the free ends 321 and the remainder of the impeller blades 316 are oriented somewhere between being substantially perpendicular to and substantially parallel to the casings 315, the driveline 307, and the central longitudinal axis 303 of the driveline 307.

In embodiments that include multiple impellers 306 (e.g., two or more), each of the impellers 306 may be oriented to have their respective impeller blades 316 (e.g., the free ends 321 of the impeller blades 316) open in any direction (e.g., at angle from 0 to 360 degrees about the X-axis). In some embodiments, a first impeller 306 may be rotationally aligned with a second impeller 306 (e.g., about the X-axis). For example, the first impeller 306 and the second impeller 306, may each be oriented to have their respective impeller blades 316 (e.g., the free ends 321 of the impeller blades 316) open in the X-Y plane. In additional embodiments, a first impeller 306 may be rotationally offset from a second impeller 306 (e.g., about the X-axis). For example, the first impeller 306 may be oriented to have its impeller blades 316 (e.g., the free ends 321 of the impeller blades 316) open in the X-Y plane, and the second impeller 306 may be oriented to have its impeller blades 316 (e.g., the free ends 321 of the impeller blades 316) open in the X-Z plane. In operation, the one or more impellers 306 may be capable of achieving about 4.5 liters per minute of blood flow (estimated) at a rotational speed of about 3000 RPM.

In some embodiments, the impellers 306 may transition between the deployed state, the partially deployed state, and the stowed state by axially translating the impellers 306 (e.g., the casings 315 and the impeller blades 316) relative to the driveline 307. For example, the driveline 307 may include an internal rod portion that extends through the casings 315. The internal rod that interacts with cam portions at the fixed ends 319 of the impeller blades 316 to open the impeller blades 316. Axial translation of the casings 315 (e.g., toward the distal end 102) relative to the driveline 307 may open the impeller blades 316, and axial translation of the casings 315 (e.g., toward the proximal end 304) in the opposite direction relative to the driveline 307 may stow the impeller blades 316.

In additional embodiments, the impellers 306 (e.g., the impeller blades 316) may comprise shape changing materials such that the impellers 306 (e.g., the impeller blades 316) are configured to change shapes in one or more various ways. For example, the impellers 306 (e.g., the impeller blades 316) may be configured to selectively move between the deployed state, the deployed state, and the partially deployed state. In some embodiments, the impellers 306 (e.g., the impeller blades 316) may be configured to rotate or twist to selectively vary the pitch of the impeller blades 316. In additional embodiments, the impeller blades 316 may be configured to bend to selectively alter the curvature of the impeller blades 316. Accordingly, the impellers 306 (e.g., the casings 315 and the impeller blades 316) may substantially free of any fasteners or separate mechanical mechanisms (e.g., cam mechanisms, springs, etc.).

As previously discussed with regard to FIG. 18, certain impeller shapes and curvatures can optimize blood flow and minimize hemolysis in both chronic implantable and temporary circulatory assist devices. Accordingly, the impellers 306 (e.g., the impeller blades 316) according to embodiments of the disclosure may change shape on demand to meet patient needs as they arise that can be delivered and removed without surgery. For example, the shape of the impellers 306 (e.g., the impeller blades 316) may be changed to lengthen their diameter (e.g., increase the length of the impeller blades 316) to increase flow and the impeller blades 316 can be twisted into an optimized shape to improve flow, minimize turbulence, minimize thrombosis risk, and minimize hemolysis and can be returned to original shape also on demand to facilitate removal via percutaneous catheter means, not surgery. Additionally, the length, shape, twist, curvature and/or overall form of the impeller blades 316 may be changed on demand in any of the ways previously described with reference to FIGS. 18-20.

As shown in FIG. 21, the free ends 321 of the impeller blades 316 of the impellers 306 may be configured to rotate outward from the pockets 317 of the casings 315 and toward the proximal end 304 of the wireless circulatory assist pump 300. Conversely, the free ends 321 of the impeller blades 316 of the impellers 306 may be configured to rotate and toward the distal end 302 to stow the impeller blades 316 in the pockets 317 of the casings 315. Accordingly, when the wireless circulatory assist pump 300 is withdrawn into the insertion and removal catheter 330 to collapse the wireless circulatory assist pump 300, the axial movement of the insertion and removal catheter 330 is aligned with the direction to stow the impeller blades 316 within the casings 315 of the impellers 306.

At the proximal end 304, the wireless circulatory assist pump 300 includes a small portion 327 at the most proximal end, and a large portion 329 connected to the small portion 327. The small portion 327 may be separated from the large portion 329 by an abrupt ledge at which a size (e.g., diameter) of the proximal end 304 increases from a size (e.g., diameter) of the small portion 327 to the size (e.g., diameter) of the large portion 329. The small portion 327 may additionally define an opening (e.g., cavity or recess) within the proximal most end. For example, the opening defined by the proximal most end of the small portion 327 may exhibit a tapered shape with the opening gradually getting smaller from the proximal most end toward the distal end 302, and may terminate within the small portion 327 proximate the large portion 329 of the proximal end 304.

The small portion 327 may be configured to removably connect to (e.g., receive) at least a portion of the insertion and removal catheter 330. For example, the wireless circulatory assist pump 300 may be configured to be connected to an end of the insertion and removal catheter 330 and may be secured to the insertion and removal catheter 330 during insertion and removal within the subject's blood vessel(s) (e.g., the aorta).

The insertion and removal catheter 330 may include an outer sheath 332, an intermediate sheath 333, and an inner sheath 334. The outer sheath 332, the intermediate sheath 333, and/or the inner sheath 334 may include an interior lining comprising expanded polytetrafluoroethylene (ePTFE).

Within the inner sheath 334, the insertion and removal catheter 330 may additionally include one or more fingers 336 (four shown) surrounding an inner member 337 (e.g., a pin). The outer sheath 332 may be configured to move axially relative to the intermediate sheath 333, and/or the inner sheath 334. Furthermore, the fingers 336 and the inner member 337 may be configured to move axially relative to each of the outer sheath 332, the intermediate sheath 333, and the inner sheath 334.

When the inner member 337 and the fingers 336 are extended out of the outer sheath 332, the intermediate sheath 333, and the inner sheath 334, the tips of the fingers 336 may be biased radially outward and apart from one another. Each finger 336 may be spaced sufficiently apart that the small portion 327 of the wireless circulatory assist pump 300 may freely pass between the fingers 336. Furthermore, the opening defined within the proximal most end of the small portion 327 may receive the inner member 337 of the insertion and removal catheter 330, and the tapered profile of the opening may guide the insertion and removal catheter 330 to the axial center of the wireless circulatory assist pump 300. Accordingly, the proximal end 304 of the wireless circulatory assist pump 300 may be connected to and aligned with the insertion and removal catheter 330.

The outer sheath 332 may then be extended over the fingers 336 resting on the small portion 327 of the wireless circulatory assist pump 300. As the outer sheath 332 extends over the fingers 336, the outer sheath 332 may force the tips of the fingers 336 radially inward to secured the insertion and removal catheter 330 to the proximal end 304 (e.g., docking end) of the circulatory assist pump 100, and inhibit (e.g., prevent) movement of the proximal end 304 relative to the insertion and removal catheter 330. The impeller blades 316 of the one or more impellers 306 may be placed into a stowed position and the stent cage 308 may be retracted. In some embodiments the impeller blades 316 and the stent cage 308 may be withdrawn into the outer sheath 332. For example, embodiments that include impellers 306 (e.g., impeller blades 316 and/or casings 315) and/or a driveline 307 made of shape memory material, the impeller blades 316 may exhibit sufficient flexibility to naturally fold and conform within the pockets 317 of the casings 315 as the outer sheath 332 moves (e.g., axially) to encompass the stent cage 308, driveline 307, impellers 306, and the proximal end 304 of the wireless circulatory assist pump 300.

The outer sheath 332 may continue to move axially relative to the circulatory assist pump 300 until an edge of the outer sheath 332 abuts the motor 314 and/or the canister in which the motor 314 is enclosed, and the circulatory assist system 301 is in the collapsed state (shown in FIG. 22).

FIG. 22 depicts the wireless circulatory assist pump of FIG. 21 in the collapsed state, according to embodiments of the present disclosure. Referring now to FIG. 22, when the circulatory assist system 301 is in the collapsed state, the stent cage 308, impellers 306, driveline 307, and proximal end 304 of the wireless circulatory assist pump 300 are all encompassed within the outer sheath 332. The outer edge of the outer sheath 332 may abut the edge of the motor 314, or alternatively, the canister encompassing the motor 314, the battery 310, and/or the circuitry 312.

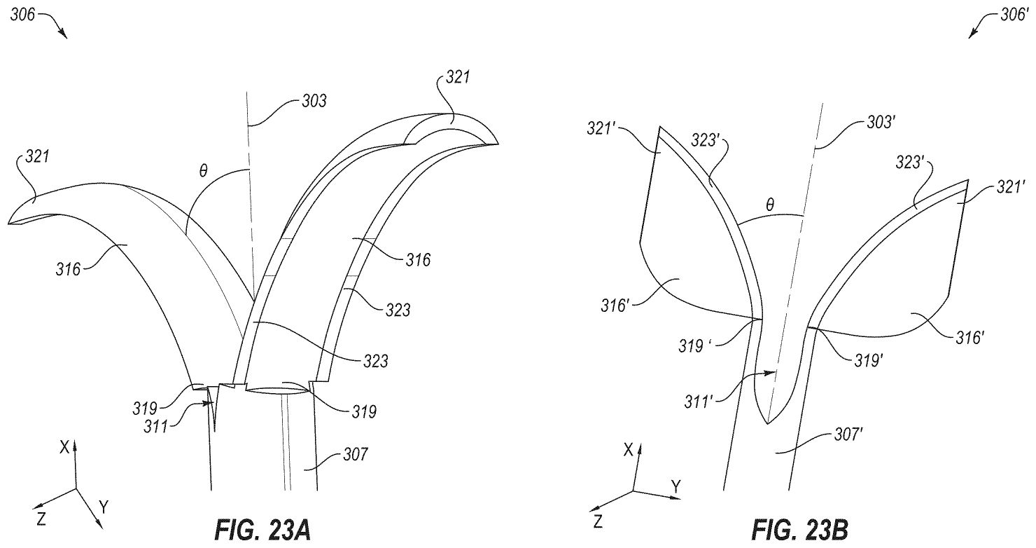

FIG. 23A depicts the impeller blades 316 of one of the impellers 306 of the wireless circulatory assist pump 300 of FIG. 21. The impeller blades 316 may be secured to (e.g., fastened to or formed integrally with) the driveline 307. For example, as shown in FIG. 23A, the driveline 307 may be separated (e.g., cut) to form a slit 311, and the separated portions of the driveline 307 may be the impeller blades 316. The fixed ends 319 of the impeller blades 316 are secured to the driveline 307, and the free ends 321 of the impeller blades 316 are free to move (e.g., rotate) relative to the fixed ends 319. Thus, in some embodiments, the impeller blades 316 may be formed integrally with the driveline 307. In additional embodiments, the impeller blades 316 may comprise a first material (e.g., a shape memory material, such as Nitinol), and the driveline 307 may comprise a second material (e.g., a metal, such as stainless steel). The impeller blades 316 may be secured to the driveline 307 by a material fusing process, such as welding.

The impeller blades 316 may be manipulated to form any desired shape for circulating blood flow within the patient while also reducing the risk of hemolysis. In some embodiments, the impeller blades 316 may exhibit rounded shapes. For example, the impeller blades 316 may be curved or arcuate from the fixed ends 319 to the free ends 321 in at least one plane (e.g., the X-Y plane). Additionally, the profile of the impeller blades 316 may also be rounded. For example, as shown in FIG. 23A, the impeller blades 316 may exhibit a concave surface (e.g., outer surface) and a convex surface (e.g., inner surface) opposite the concave surface.

In embodiments in which the impeller blades 316 comprise a shape memory material, the impeller 306 may be configured to open to a predetermined “remembered” state in which the impeller blades 316 oriented in the deployed state (e.g., perpendicular to the driveline 307) or in a partially deployed state between the deployed state and the stowed state. More specifically, the impeller 306 may be configured to open to a predetermined “remembered” state in which the impeller blades 316 are oriented at an angle (θ). The angle (θ) is an angle of the impeller blades 316 relative to the central longitudinal axis 303 of the impeller 306. For example, in embodiments in which the impeller blades 316 exhibit a planar shape, the angle (θ) may be measured at any point along a length of the impeller blades 316. In embodiments in which the impeller blades 316 are curved, the angle (θ) may be an average of an angle that includes a first component measured from the central longitudinal axis 303 to free ends 321 of the impeller blades 316, and a second component measured from proximate the fixed end 319 of the impeller blades 316. Accordingly, for the impeller blades 316 shown in FIG. 23A, the free ends 321 are oriented at about a 90° angle relative to the central longitudinal axis 303, and the fixed ends 319 are oriented at about a 10° angle relative to the central longitudinal axis 303. Accordingly, the angle (θ) of the impeller blades 316 shown is about 50°. In some embodiments, the angle (θ) may be any angle within a range of from about 1° to about 135°, such as from about 5° to about 120°, from about 15° to about 90°, such as from about 30° to about 60° (e.g., about 45°).

In embodiments in which the impeller blades 316 comprise shape memory material, the impeller 306 may include a predetermined “remembered” state in which the convex surfaces of the impeller blades 316 are oriented axially toward the distal end 302 of the circulatory assist pump 300. In additional embodiments in which the impeller blades 316 comprise shape memory material, the impeller 306 may include a predetermined “remembered” state in which the concave surfaces of the impeller blades 316 are oriented axially toward the distal end 302 of the circulatory assist pump 300. Accordingly, when the impeller blades 316 are in operation to facilitate blood flow within the subject's aorta, the rounded (curved) portions of the blades may be oriented upstream, which may reduce hemolysis.

In some embodiments, the impeller blades 316 include exterior frames 323 and may be made of substantially similar materials, exhibit substantially similar properties, and exhibit substantially similar functionality to the shape change impeller 106A of FIGS. 19-20B.

FIG. 23B depicts another embodiment of impeller blades 316′ of a wireless circulatory assist pump (e.g., the wireless circulatory assist pump 100, 300), according to embodiments of the present disclosure. In FIG. 23B, and the associated description, functionally similar features (e.g., structures, materials) to those of the impeller 306 of FIG. 23A are referred to with similar reference numerals including a prime (′) following the reference numerals. To avoid repetition, not all features shown in FIG. 23B are described in detail herein. Rather, unless described otherwise below, a feature as shown in FIG. 23B including a prime (′) following the reference numeral of FIG. 23A will be understood to be substantially similar to the previously described feature of FIG. 23A.

Referring now to FIG. 23B, the impeller blades 316′ may be secured to (e.g., fastened to or formed integrally with) the driveline 307′. For example, as shown in FIG. 23B, the driveline 307′ may be separated (e.g., cut) to form a slit 311′, and the separated portions of the driveline 307′ may form frames 323′ of the impeller blades 316′. The frames 323′ may be made of substantially similar materials, exhibit substantially similar properties, and exhibit substantially similar functionality to the shape change impeller 106A of FIGS. 19-20.

The slit 311′ between the impeller blades 316′ may be larger than the slit 311 of FIG. 23A. In addition, the impeller blades 316′ may include additional material (e.g., shape memory material) connected to the frames 323′. As shown in FIG. 23B, each of the impeller blades 316′ may exhibit the shape of a butterfly wing. For example, the impeller blades 316′ may exhibit a curved triangular shape, in which the frames 323′ extend axially and radially away from the driveline 307′. Although illustrated as having sharp edges, the frames 323′ and the remainder of the impeller blades 316′ may exhibit rounded shapes, which may reduce hemolysis during operation.

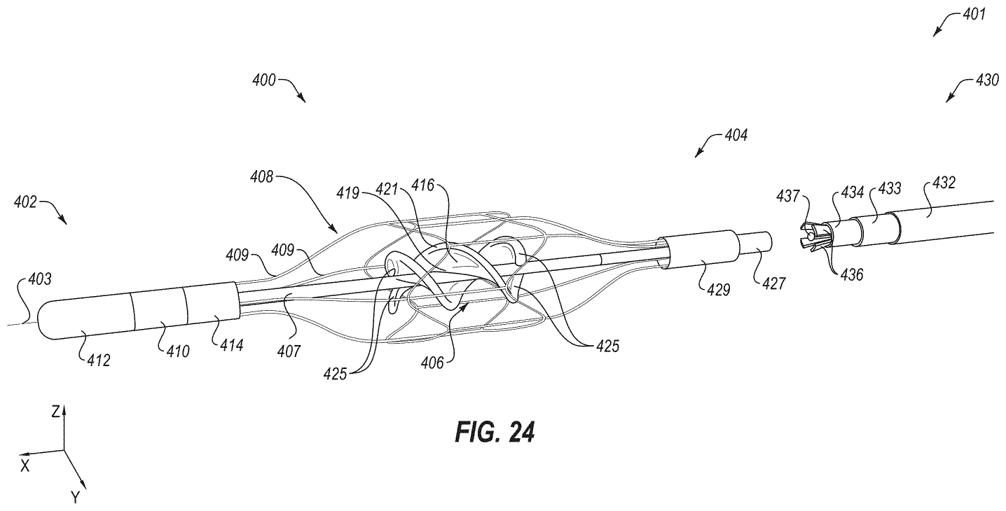

FIG. 24 depicts a circulatory assist system 401 that includes a wireless circulatory assist pump 400 and an insertion and removal catheter 430, in accordance with embodiments of the present disclosure. In FIG. 24, and the associated description, functionally similar features (e.g., structures, materials) to those of the wireless circulatory assist pump 300 of FIG. 21 are referred to with similar reference numerals incremented by 100. To avoid repetition, not all features shown in FIG. 24 are described in detail herein. Rather, unless described otherwise below, a feature as shown in FIG. 24 designated by a reference numeral that is a 100 increment of the reference numeral of FIG. 21 will be understood to be substantially similar to the previously described feature of FIG. 21.

Referring now to FIG. 24, the wireless circulatory assist pump 400 is shown in an expanded state. The wireless circulatory assist pump 400 generally includes a distal end 402, a proximal end 404 (e.g., docking end), and an impeller 406 enclosed within a stent cage 408 between the distal end 402 and the proximal end 404. The wireless circulatory assist pump 400 may further include a battery 410, circuitry 412 and a motor 414. The circuitry 412 may comprise a wireless charging circuit, a communications circuit, and a control circuit. As shown in FIG. 24, the battery 410, the circuitry 412, and the motor 414, may all be located at or near the distal end 402, but it will be understood that one or more, or all, of the battery 410, the wireless charging circuit, the communications circuit, the control circuit, and the motor 414, may alternatively be located at or near the proximal end 404.

The wireless circulatory assist pump 400 may include a single impeller 406 arranged on a driveline 407. For example, the impeller 406 may be formed integrally with the driveline 407 such that a central portion of the impeller 406 is the driveline 407. In additional embodiments, the wireless circulatory assist pump 400 may include multiple (e.g., two or more) impellers 406 arranged in series on a common driveline 407. Accordingly, one revolution of the driveline 407 may correspond to one revolution of the impeller 406. The impeller 406 includes impeller blade 416 configured to move (e.g., radially) relative to the driveline 407 and the central longitudinal axis 403 of the impeller 406. The impeller blade 416 may be configured to move radially from a deployed state (e.g., expanded or open state), as shown, to a stowed state (e.g., collapsed or closed state) (see, e.g., FIG. 22), and vice versa. The impeller blade 416 may additionally be configured to move to states between the deployed state and the stowed state (e.g., to a partially deployed state, also referred to as a partially expanded state or partially open state).

The impeller blade 416 may exhibit a generally helical shape (e.g., an auger shape), including a fixed end 419 (e.g., fixed edge), a free end 421 (e.g., radially outermost edge or free edge), and an interior material connected to an interposed between the free end 421 (e.g., the exterior frame) and the fixed end 419. The free end 421 of the impeller blade 416 may be reinforced relative to the fixed end 419. Additionally, the free end 421 may exhibit a greater thickness than the fixed end 419 of the impeller blade 416. Additionally, the impeller 406 may include reinforced ends 425 at distal and proximal ends of the impeller blade 416. Having a greater thickness at the free end 421 of the impeller blade 416, and reinforced ends 425 may improve the resilience of the impeller blade 416, and may help the impeller blade 416 retain its helical shape while being used to facilitate blood circulation within the subject's aorta. In operation, the impeller blade 416 (e.g., the helical impeller blade) may be capable of achieving about 4.5 liters per minute of blood flow (estimated) at a rotational speed of about 4,500 RPM.

In additional embodiments, the impeller 406 may comprise shape changing material such that the impeller 406 is configured to change shapes in one or more various ways. For example, the impeller 406 may be configured to selectively move between the deployed state, the deployed state, and the partially deployed state. In some embodiments, the impeller 406 (e.g., the impeller blade 416) may be configured to rotate or twist to selectively vary the pitch of the impeller blade 416. For example, the impeller blade 416 may be configured to expand or contract along the central longitudinal axis 403 of the impeller 406. In additional embodiments, the impeller blade 416 may be configured to bend to selectively alter the curvature of the impeller blade 416. Accordingly, the impeller 406 may substantially free of any fasteners or separate mechanical mechanisms (e.g., cam mechanisms, springs, etc.).

As previously discussed with regard to FIG. 18, certain impeller shapes and curvatures can optimize blood flow and minimize hemolysis in both chronic implantable and temporary circulatory assist devices. Accordingly, the impeller 406 (e.g., the impeller blade 416) according to embodiments of the disclosure may change shape on demand to meet patient needs as they arise that can be delivered and removed without surgery. For example, the shape of the impeller 406 (e.g., the impeller blade 416) may be changed to lengthen its diameter (e.g., increase the radius of the impeller blade 416) to increase flow, and the impeller blade 416 can be twisted into an optimized shape to improve flow, minimize turbulence, minimize thrombosis risk, and minimize hemolysis and can be returned to original shape also on demand to facilitate removal via percutaneous catheter means, not surgery. Additionally, the length, shape, twist, curvature and/or overall form of the impeller blade 416 may be changed on demand in any of the ways previously described with reference to FIGS. 18, 19, 20A, and 20B.

As previously discussed, the free end 421 of the impeller blade 416 of the impeller 406 may be configured to expand radially outward from the central longitudinal axis 403 of the impeller 406, and collapse radially inward toward the central longitudinal axis 403. Accordingly, when the wireless circulatory assist pump 400 is withdrawn into an insertion and removal catheter (e.g., the insertion and removal catheter 330), the axial movement of the insertion and removal catheter collapses the stent cage 408 cage and the impeller 406 into the stowed position.

In certain embodiments, the belt, which is to be worn by the patient (see, e.g., FIG. 12), is used to control the pulsatile cuff pulsations, provides wireless power to the lower aortic stent impeller, provides, e.g., vibrational harmonic resonant vibrations or other energy to prevent blood clot formation(s) at, e.g., high risk stagnation points, magnetically or by sound wave pulsations grabs blood and moves it with electro-magnetic or sound waves (may reduce 1,500 RPM to reach 4.5 liters flow to 1,000 RPM estimated), and delivers bioelectric signals into tissues and the aorta releasing proteins beneficial to organ and whole body health (note pulsatility also promotes release of beneficial organ health proteins from the aorta and other arteries and tissues).

The removable pulsatile cuff stent may be placed just above the lower impeller aortic stent, which achieves approximately 2 liters per minute flow improvement on its own. The removable pulsatile cuff stent can be designed to push blood up and down or just down by programming the pulsatile elements. The removable pulsatile cuff stent is timed to pulse squeeze in optimization with the heart natural pulsatility. When the pulsatile cuff stent is in place pulsating, the impeller RPM may be reduced to 1,500 RPM to reach 4.5 liters per minute flow (estimated). This cuff placement provides the option for pulsatile flow circulatory assist augmentation.

Pulsatile stent grafts (see, e.g., FIG. 8) are disclosed in, e.g., Palma et al. “Pulsatile stent graft: a new alternative in chronic ventricular assistance,” Revista Brasileira de Cirurgia Cardiovascular (2013), 28(2):217; dx.doi.org/10.5935/1678-9741.20130031, the contents of each of which are incorporated herein by this reference.

In one embodiment, a pulsatile stent graft may be included within the system, placed mid-aorta, while substantially continuous impeller power is applied in the bare aortic stent in the lower aorta.

Preferred such systems for use herein are described in: Pahlevan and Gharib “A wave dynamics criterion for optimization of mammalian cardiovascular system,” J. Biomech. 2014 May 7; 47(7):1727-32. doi: 10.1016/j.jbiomech.2014.02.014. Epub 2014 Feb. 20, Pahlevan and Gharib “A Bio-Inspired Approach for the Reduction of Left Ventricular Workload,” PLOSone, (Jan. 24, 2014); https://doi.org/10.1371fjournal.pone.0087122, Pahlevan and Gharib “Aortic Wave Dynamics and Its Influence on Left Ventricular Workload,” PLOSone, (Aug. 11, 2011); https://doi.org/10.1371fjournal.pone.0023106, U.S. Pat. No. 9,125,655 to Gharib et al. (Sep. 8, 2015) for Correction and Optimization of Wave Reflection in Blood Vessels; U.S. Pat. No. 7,998,190 to Gharib et al. (Aug. 16, 2011) for Intravascular Miniature Stent Pump; U.S. Pat. No. 7,163,385 to Gharib et al. (Jan. 16, 2007) for Hydroimpedance Pump; U.S. Pat. No. 8,092,365 to Rinderknecht et al. (Jan. 10, 2012) for Resonant Multilayer Impedance Pump; U.S. Pat. No. 7,883,325 to Kheradvar et al. (Feb. 8, 2011) for Helically Actuated Positive-Displacement Pump and Method and U.S. Pat. No. 9,125,655 B2 to Phalevan, the contents of each of which are incorporated herein by this reference.

Preferably, the pulsating cuff pump is positioned in the upper aorta of the subject above the stent cage impeller, which is positioned lower in the aorta. Preferably, two aortic stents in series in the aorta, the top aortic stent being fully pulsatile and the bottom aortic stent semi-pulsatile (meaning it turns, but it turns so far away from heart that it does not take away pulsatility, it just accelerates it). This relative positioning of the two pumps maximizes flow while minimizing impeller RPM. The combination of the pulsating cuff aortic stent graft in the upper aorta with the impeller pump/aortic stent in the lower aorta reduces RPMs from, e.g., 4,500 rpm to attain 4.5 liters per minute flow to 1,500 rpm, and provides advantages in terms of hemodynamics, expression of protein(s), and flow not found in either device alone. Less RPMs requires less power, which translates to a system that is easier to power wirelessly. There is also less of a risk of a mechanical breakdown, and less resulting damage to blood cells from hemolysis.

Such a system, may be combined with, e.g., a vibrating harmonic resonant device to reduce and hopefully prevent blood clots, which is “the Achilles' heel” of chronic implants. A harmonic resonant vibration system to reduce blood clots in such a system is described in U.S. Publication No. US 20190125932 A1 to Leonhardt et al. (May 2, 2019) for “Preventing Blood Clot Formation, Calcification and/or Plaque Formation on Blood Contact Surface(s),” the contents of which are incorporated herein by this reference. The system may also (or alternatively) utilize an electric charge surface treatment of the implant to further reduce risk of blood clots, calcification, and plaque forming on the device.

In certain embodiments, the system includes a bi-layer magnetic fluid graft that further increases flow without hemolysis (e.g., the system utilizes a magnetic fluid-filled silicon (bi-layer) graft liner placed on the inside of the impeller stent) where the pulsating waves augment aortic flow.

In certain embodiments, the system magnetically “grabs” blood via iron particles in blood and manages flow wave pulses to optimization and flow optimization timing, which further enhances flow without increasing hemolysis. For example, pulsed electromagnetic waves cam be utilized to “grab” the iron in the patient's blood and move it in waves via an external belt.

The system can further include bioelectric coils on the stent to control expression and/or release of protein(s) such as those that build strength of aortic muscle and/or aid in kidney recovery. See, e.g., the earlier incorporated U.S. Patent publication US 2017/0266371 A1 to Leonhardt et al. (Sep. 21, 2017) and/or Macfelda et al. “Bioelectrical signals improve cardiac function and modify gene expression of extracellular matrix components” ESC Heart Failure 2017; 4: 291-300 (published online 30 Jun. 2017); DOI: 10.1002/ehf2.12169, the contents of which are incorporated herein by this reference. Via the system, inflammation and blood pressure can be managed with bioelectric signal protein expressions and membrane potential management. The platform can also be used to aid in the creation and control of smooth muscle formation in the aorta.

In certain embodiments, wireless powered and programmed micro coils are utilized with the system to control aortic tissue protein expressions and to increase smooth muscle mass and to control pulsations of natural aortic muscle, a cellular muscle-based “second heart.” For example, pacing the timed electrical pulse signals may be utilized to trigger contractions of smooth muscle so to make the natural aorta a beating “second heart” optimized with native pulsatile flow.

The wireless powered and programmed micro coils can be further used to control chronic inflammation and blood pressure with real time reads and adjustments.

The system itself preferably utilizes programmed, real-time optimization to manage flow, hemolysis, power, and patient hemodynamics real time. The programming can be configured to change parameters, e.g., with the subject's exercise, sleep, heart failure conditions, etc., including monitoring fluid level in the patient's lungs, etc.

In certain embodiments, the system includes vibrational harmonic resonant tuned technology, which reduces risk of thrombosis (blood clot formations), reduces risk of plaque or calcification formations, increases gas exchanges in aorta, and promotes healthy protein release in aorta. It is relatively easily mounted into the same belt providing wireless power and controlling pulsating implants and micro coils. Including micro coils controls protein expression in the aorta to, e.g., increase elasticity, control blood pressure, improve organ health, and control inflammation.

Blood clots have been the “Achilles heel” of many other chronic implant devices. Resonant harmonic vibrational energy technology may be utilized to reduce the risk of this problem. Tuned harmonic resonant vibration may be used to prevent blood clot formation at high risk stagnation points on the device. The harmonic resonance for each high risk stagnation point may be individually customized and stored in a microprocessor. The vibrational energy may be delivered in pulses in a loop hitting each high risk location of the device to prevent a large accumulation of a blood clot, which might develop.

Pulsatility results in healthier hemodynamics, less risk of thrombosis, together with cellular arterial wall protein expression for superior organ recovery and patient well-being. The device described herein combines the best of pulsatile flow with continuous flow. Using pulsatile and continuous flow optimizes hemodynamics and lessens the risk of thrombosis.

In certain embodiments, the system utilizes a motor console for precision performance and low vibration, with flushing built in.

In certain embodiments, BION micro coil implants are incorporated into the system. They may be utilized to release proteins for the heart, aorta, arteries, lungs and kidney health. They may also be utilized to provide real time data on performance, flow, pressures etc.

The system can be utilized variously. For instance, as a temporary catheter alone for 6 to 72 hours. As a temporary catheter with removable pulsating cuff stent in series with both removed after use of 6 to 72 hours. The temporary use catheter may be removed, but the pulsating cuff stent may be left in place for chronic long term use. The catheter and drive shaft can be disconnected from the impeller stent, which can then be switched to wireless power on a standalone basis.

In certain embodiments, there are two aortic stent based circulatory assist pumps in series in the aorta, one upper and one lower, the upper one being pulsatile.

In certain embodiments, the impeller stent can be left out/removed, and the pulsating aortic cuff stent left in place.

The device may be removed should the need for the device abate (e.g., upon recovery of the patient). For removing the device, a modified Seldinger technique (or comparable technique) can be applied in reverse utilizing a catheter that interacts with, e.g., the pump for removal. The impeller blades may first be retracted and the stent cage then collapsed about it to reduce the cross-sectional diameter of the pump to aid in removal.

The foregoing can be supported with a vibrational harmonic resonance technology for preventing blood clot formations (thrombosis), but this is especially preferred when the system is used for chronic implant use. Furthermore, the foregoing can be supported with the release of bioelectrically controlled release of protein(s) from, e.g., the aorta, tissues, and arteries to assist in healing. Further, the foregoing can be supported by electromagnetic wave or sound wave pulsations to further enhance blood flow improvement.

Although it is an advantage of the device to not need to cross the aortic valve, in certain embodiments, the described encaged pump system may be combined advantageously with a device that does cross the aortic valve (e.g., in high head/low flow applications). Such a system includes placement of the device that does cross the aortic valve at the tip of the catheter, beyond the aortic valve and placement of the herein described second device encaged impeller (bare aortic stent and pump on the catheter) proximal the renal arteries that feed the kidneys. The first such pump may be a second of the herein described pumps or a pump akin to the HeartMate PHP percutaneous heart pump. The second such pump may be that of FIG. 11 adapted by extending the drive shaft further to interact and drive the first pump. The two pumps are placed on the same catheter and may utilize the same drive shaft. The first pump operating high near the heart (for left ventricle unloading) past the heart valve and the second pump in positioned in the lower aorta, just above the renal arteries (for renal output improvement), i.e., the second pump in the mid to lower stomach and the first pump up in the upper mid chest (usually 20 to 30 cm in most people).

In such a situation, sometimes the required operating conditions for a patient are beyond the reach of a single, standard pump, and it is best to combine simple pump performances that add up to the necessary requirements. Positioning pumps in series as described herein, or connected along a single line, allows the system to add the head from each pump together to meet the high head, low flow system requirements. This is because the fluid pressure increases as the continuous flow passes through each pump, much like how a multi-stage pump works. For example, if two of the same pumps are in series, the combined performance curve will have double the head of a single pump for a given flow rate. For two different pumps, the head is still added together on the combined pump curve, but the curve will most likely have a piecewise discontinuity.

In situations where a high, constant pressure is required, speed control may need to be included with, for example, the first pump in such a system. This configuration achieves the high pressure that is needed, while keeping a low flow, because the fixed-speed pump feeds into the speed-controlled pump, which adjusts its output with a pressure transmitter to add only enough head to maintain a constant pressure. This device would combine the benefits of both designs in one product. Having two in series reduces RPMs needed for both to get same flow improvement.

The disclosure is further described with the aid of the following Examples.

### Example I

A prior art IMPELLA 2.5® heart pump (Abiomed) pulls blood from the left ventricle through an inlet area near the tip and expels blood from the catheter into the ascending aorta. The IMPELLA 2.5® heart pump is designed to temporarily (≤6 hours) protect the patient hemodynamically during a high-risk procedure (e.g., in patients experiencing: advanced heart failure, cardiogenic shock, and/or post-cardiotomy cardiogenic shock). The IMPELLA 2.5® device is inserted into a patient via a standard catheterization procedure through the femoral artery, into the ascending aorta, across the valve and into the left ventricle. The IMPELLA 2.5® device is thought to stabilize hemodynamics, unloads the left ventricle, perfuses the end organs, and allows for recovery of the native heart.

The IMPELLA 2.5® device spins at approximately 50,000 RPM with flows of 2.5 l/min on the highest possible setting. Reportedly, Abiomed's 5.0 device spins at 33,000 RPM with maximum flows of 5.3 l/min on the highest possible setting.

The IMPELLA 2.5® device needs 55,000 RPMs (turns of impeller) to achieve 4.5 liters per minute flow at the level of the renal arteries for cardio-renal dysfunction recovery.

Utilizing the device of FIG. 7, 4.5 liters flow at the level of the renal arteries (goal is to increase renal output and recovery) were achieved in a pig with only 4500 RPMs. Lower RPMs results in less damage to blood cells (hemolysis), less heat, less wear, less risk of mechanical breakdown, and less power needs.

The device of FIG. 7 is wireless powered when combined with a second pulsating cuff stent higher in the aorta achieves 4.5 liters flow with only 1,500 RPMs, and may be left in the patient up to 5 years. The IMPELLA 2.5® device is to be removed in 72 hours and is connected by a drive shaft to an external motor.

FIG. 9 is a picture of a device comprising the impeller and surrounding stent cage implanted and actuated within a pig cadaver.

The IMPELLA 2.5® device needs to spin its impellers at 18,500 to 50,000 RPM to reach 4.5 liters per minute flow through the device, which increases risk of hemolysis and mechanical breakdown. The IMPELLA 2.5® device does not reach 4.5 liters per minute true flow in the patients with these RPMs, only these flow rates through the small orifices of the associated small diameter catheters. The actual patient flow improvement is less than ½ this device flow rate, i.e., under 2.25 liters per minute patient flow improvement.

In certain embodiments, the device utilizes strong radial force deployment to maintain its position in the aorta and occupies nearly all (or all) of the entire inner diameter of the subject's aorta, and thus the 4.5 liters per minute flow through device is also 4.5 liters per minute flow improvement for the patient. The strong radial force utilized in the system limits repositioning of the device. Occupying this much of the aorta allows for the use of the relatively lower rpm of the device.

Wireless power, which powers the device of FIG. 7, results in a higher quality of life for patients. The patient can go home, with less risk of infection and less risk of movement of position.

### Example II

The herein described circulatory assist device is combined with a heart regeneration bioelectric stimulator, micro infusion pump, and mixed composition for implantation into a subject's aorta as described herein. In such a combination, the circulatory assist pump off loads work load from the heart, thus improving perfusion to improve regeneration results. The subject's heart recovers over time.

Expression of desirable protein(s) may be accomplished via, e.g., implanted micro coils on the stent. See, e.g., the earlier incorporated U.S. Patent publication US 2017/0266371 A1 to Leonhardt et al. (Sep. 21, 2017) and/or the earlier incorporated Macfelda et al. “Bioelectrical signals improve cardiac function and modify gene expression of extracellular matrix components” ESC Heart Failure 2017; 4: 291-300 (published online 30 Jun. 2017).

As previously described, such micro coils too can utilize wireless energy. Wireless control extends to pulsatility, speed, and/or impeller angle of the various components of the system.

### Example III

As depicted in FIG. 13, a physiologically accurate mock circulation loop (static mock flow loop) is used to test the devices at the Cardiovascular Innovation Institute in Louisville, Ky. FIG. 13 shows the dynamic mock flow loop includes (a) a left ventricle, (b) a left ventricular assist device (LVAD), (c) systemic compliance, (d) venous reservoir, and (e) atrial elements. These mock flow loops quantify the hydraulic and hemodynamic performance of the LVAD.

### Example IV

A wireless circulatory assist pump 100 (FIG. 18) is made and encaged within a stent cage 108 (FIG. 18) between the distal end 102 and the proximal end 104. The impeller blades 116 each have a length of about 8 mm from the fixed end 119 to the free end 121 and are made of 17-4 PH stainless steel.

The encaged circulatory assist pump (FIG. 18) is placed in the subject's aorta just above the renal arteries. The stent cage is expanded and the impeller blades extended within the aorta.

The impeller blades are set to rotate at 7,500 rpm in a 20 mm aorta distended with stent radial force to 22 mm, thus producing an increase of 1.5 liters per minute flow from a starting base of 3.5 liters per minute increasing to 5.0 liters per minute total flow in the aorta just above the renal arteries. Dependent on, for example, the patient being treated, an optimal pump speed can be as high as 10,000 rpm.

Computational fluid dynamics testing is conducted used to determine flow rates (particularly flow into the renal arteries), aortic pressure differential, and coronary flow rates, and thus brain and hemolysis risk.

### Example V

A circulatory assist pump is made and encaged within a stent cage. The impeller blades have an impeller diameter of 13.5 mm long from tip to tip and are made of 17-4 PH stainless steel.

The impeller blades are set to rotate at 7,500 rpm in an open stent (outer diameter) aorta distended of 22.86 mm.

The boundary conditions are as follows:

Flow Inlet (L/min) of 3.5, 4.5, and 5.5.

Impeller speeds (rpm) of 7,500, 10,500, and 15,000.

### Example VI

An upper aortic pulsating stent graft useful herein has the following dimensions and specifications:

Outer Diameter of 24 mm aortic stent for being placed, e.g., in a 20-22 mm aorta

Total Length of 6 cm before placement in the 22 mm aorta (lengthens when compressed).

Hoop Strength of 15.8 N/cm

Radial Resistance Force of 1.27 N/cm

Chronic Outward Force of 0.31 N/cm

Three (3) pulsating wireless powered bands each 1.5 cm wide each wrapped around stent. Only one pulsates at any given time.

Aortic stent is ¾'s covered in ePTFE (expanded polytetrafluoroethylene) matching with positions of pulsatile bands.

Each pulse band on each pulsation moves covered stent inward into the aorta 3 mm (a 3 mm aortic pulse wave).

Pulsation is time matched to natural pulses of the subject's heart (e.g., “native flow”) with a slight time delay for time for pulsed blood flow to reach the aorta.

### Example VII

Powering an impeller pump positioned within a stent cage of FIG. 7 was successfully demonstrated by the Queensland University of Technology (QUT) in Brisbane, Australia, using the QUT wireless power system. An AC/DC power supply providing 1.6 volts connected to a transmitter coil to a series capacitor coil and inverter (set at 1 megahertz) and controller. The system was about 1.3 Watt.

### REFERENCES

The contents of each of the following references are incorporated herein by this reference:
Novel electro-active shape memory polymers for soft actuators, YingJun An and Hidenori Okuzaki 2020 Jpn. J. Appl. Phys. 59 061002.
Review of electro-active shape-memory polymer composite, YanjuLiu, HaibaoLv, XinLan, JinsongLeng, ShanyiDu, Composites Science and Technology, Volume 69, Issue 13, October 2009, Pages 2064-2068.
Electro and Light-Active Actuators Based on Reversible Shape-Memory Polymer Composites with Segregated Conductive Networks, Zhao Xu, Chao Ding, Dun-Wen Wei, Rui-Ying Bao, Kai Ke, Zhengying Liu, Ming-Bo Yang, and Wei Yang, ACS Applied Materials & Interfaces 2019 11 (33), 30332-30340.
Electroactive shape memory polymer based on optimized multi-walled carbon nanotubes/polyvinyl alcohol nanocomposites, Fei-PengDu, En-ZhouYe, WenYang, Tian-HanShen, Chak-YinTang, Xiao-LinXie, Xing-PingZhou, Wing-CheungLaw, Composites Part B: Engineering, Volume 68, January 2015, Pages 170-175.
Strong Electroactive Biodegradable Shape Memory Polymer Networks Based on Star-Shaped Polylactide and Aniline Trimer for Bone Tissue Engineering, Meihua Xie, Ling Wang, Juan Ge, Baolin Guo, and Peter X. Ma, ACS Applied Materials & Interfaces 2015 7(12), 6772-6781, DOI: 10.1021/acsami.5b00191.
Effect of Geometrical Changes of Impeller on Centrifugal Pump Performance, Pranit M. Patill, Shrikant B. Gawas2, Priyanka P. Pawaskar3, Dr. R. G. Todkar4, International Research Journal of Engineering and Technology (IRJET) Volume: 02 Issue: 2 May 2015.
Pump Performance Curve Shapes and How to Modify Them, Allan R. Budris, Jan. 1, 2011, https://www.waterworld.com/technologies/pumps/article/16192402/pump-performance-curve-shapes-and-how-to-modify-them.
Designing a More Effective Left Ventricular Assist Device Using CFD Simulation, Mohammad Haddadi, Jun. 29, 2018, https://www.ansys.com/blog/designing-lvad-using-simulation/.
Biologically Inspired, Open, Helicoid Impeller Design for Mechanical Circulatory Assist, Park, Jiheum; Oki, Kristi; Hesselmann, Felix; Geirsson, Arnar; Kaufmann, Tim, Bonde, Pramod, ASAIO Journal. 66(8):899-908, August 2020.
Controlled Pitch-Adjustment of Impeller Blades for an Intravascular Blood Pump, Throckmorton, Amy L.; Sciolino, Michael G.; Downs, Emily A.; Saxman, Robert S.; López-Isaza, Sergio; Moskowitz, William B., ASAIO Journal. 58(4):382-389, July/August 2012.
Dual-Propeller Cavopulmonary Pump for Assisting Patients with Hypoplastic Right Ventricle, Jagani, Jakin N.; Untaroiu, Alexandrina; Kalaria, Amit D., ASAIO Journal. 65(8):888-897, November/December 2019.
Computational Fluid Dynamics Modeling of Impeller Designs for the HeartQuest Left Ventricular Assist Device, Curtas, Anthony R.; Wood, Houston G.; Allaire, Paul E.; McDaniel, James C.; Day, Steven W.; Olsen, Don B, ASAIO Journal. 48(5):552-561, September-October 2002.
Mechanical Circulatory Support of the Right Ventricle for Adult and Pediatric Patients With Heart Failure, Chopski, Steven G.; Murad, Nohra M.; Fox, Carson S.; Stevens, Randy M, Throckmorton, Amy L., ASAIO Journal. 65(2):106-116, February 2019.
Design and Transient Computational Fluid Dynamics Study of a Continuous Axial Flow Ventricular Assist Device, Song, Xinwei; Untaroiu, Alexandrina; Wood, Houston G.; Allaire, Paul E.; Throckmorton, Amy L.; Day, Steven W.; Olsen, Donald B., ASAIO Journal. 50(3):215-224, May-June 2004.
Clinical Implications of Physiologic Flow Adjustment in Continuous-Flow Left Ventricular Assist Devices, Tchantchaleishvili, Vakhtang; Luc, Jessica G. Y.; Cohan, Caitlin M.; Phan, Kevin; Hubbert, Laila; Day, Steven W.; Massey, H Todd, ASAIO Journal. 63(3):241-250, May/June 2017.
Scaling the Low-Shear Pulsatile TORVAD for Pediatric Heart Failure, Gohean, Jeffrey R.; Larson, Erik R.; Hsi, Brian H.; Kurusz, Mark; Smalling, Richard W.; Longoria, Raul G., ASAIO Journal. 63(2):198-206, March/April 2017.
Mechanical Cavopulmonary Assist for the Univentricular Fontan Circulation Using a Novel Folding Propeller Blood Pump, Throckmorton, Amy L.; Ballman, Kimberly K.; Myers, Cynthia D.; Litwak, Kenneth N.; Frankel, Steven H.; Rodefeld, Mark D, ASAIO Journal. 53(6):734-741, November-December 2007.
Mini Hemoreliable Axial Flow LVAD With Magnetic Bearings: Part 2: Design Description, Goldowsky, Michael, ASAIO Journal. 48(1):98-100, January-February 2002.
Testing of a Centrifugal Blood Pump With a High Efficiency Hybrid Magnetic Bearing, Locke, Dennis H.; Swanson, Erik S.; Walton, James F. II; Willis, John P.; Heshmat, Hooshang, ASAIO Journal. 49(6):737-743, November-December 2003.
Long-Term Mechanical Circulatory Support System Reliability Recommendation by the National Clinical Trial Initiative Subcommittee, Lee, James, ASAIO Journal. 55(6):534-542, November-December 2009.
A Durable, Non Power Consumptive, Simple Seal for Rotary Blood Pumps, Mitamura, Yoshinori; Sekine, Kazumitsu; Asakawa, Masahiro; Yozu, Ryohei; Kawada, Shiaki; Okamoto, Eiji, ASAIO Journal. 47(4):392-396, July 2001.
HeartMate III: Pump Design for a Centrifugal LVAD with a Magnetically Levitated Rotor, Bourque, Kevin; Gernes, David B.; Loree, Howard M. II; Scott Richardson, J.; Poirier, Victor L.; Barletta, Natale; Fleischli, Andreas; Foiera, Giampiero; Gempp, Thomas M.; Schoeb, Reto; Litwak, Kenneth N.; Akimoto, Takehide; Watach, Marv J.; Litwak, Philip, ASAIO Journal. 47(4):401-405, July 2001.
Uniquely Shaped Cardiovascular Stents Enhance the Pressure Generation of Intravascular Blood Pumps, Throckmorton, Amy L; Carr, James P.; Moskowitz, William B.; Gangemi, James J.; Haggerty, Christopher M.; Yoganathan, Ajit P., The Journal of Thoracic and Cardiovascular Surgery, Vol. 144, No. 3, September 2012.
U.S. Pat. No. 8,395,300 to Aabloo et al. issued Mar. 12, 2013.
U.S. Pat. No. 7,841,976 to McBride et al. issued Nov. 30, 2010.
U.S. Pat. No. 4,283,233 to Goldstein et al. issued Aug. 11, 1981.
U.S. Patent Pub. No. 2009/0248141 to Shandas et al. Published Oct. 1, 2009.
U.S. Publication No. US 20190125932 A1 to Leonhardt et al. (published May 2, 2019) for “Preventing Blood Clot Formation, Calcification and/or Plaque Formation on Blood Contact Surface(s).”

1. A circulatory assist pump, comprising:
a stent cage being expandable and collapsible, the stent cage of a size and shape to allow a highly open blood flow when the stent cage is positioned in an expanded state within a subject's aorta, the stent cage further configured to provide a radial force against an inner wall of the subject's aorta when the stent cage is in the expanded state such that the stent cage is positionally secured to the inner wall of the subject's aorta and such that the stent cage flexes with a natural pulsatility of the subject's aorta; and
an impeller encaged by the stent cage, the impeller comprising at least one impeller blade configured to selectively change shape.

2. The circulatory assist pump of claim 1, wherein the stent cage comprises wire-like elements exhibiting rounded shapes.

3. The circulatory assist pump of claim 2, wherein the radial force is sufficient to embed an entire thickness of the wire-like elements into the inner wall of the subject's aorta such that an opening defined within the embedded wire-like elements of the stent cage is substantially the same size as the subject's aorta.

4. The circulatory assist pump of claim 1, wherein the stent cage exhibits a balance of flexibility and rigidity such that the stent cage is configured to maintain an axial position within the subject's aorta and radially flex with the natural pulsatility of the subject's aorta.

5. The circulatory assist pump of claim 1, wherein the radial force is within a range of from about 0.1 N to about 1.0 N.

6. The circulatory assist pump of claim 1, wherein the stent cage comprises a temperature sensitive shape memory material.

7. The circulatory assist pump of claim 1, wherein the at least one impeller blade comprises a temperature sensitive shape memory material.

8. The circulatory assist pump of claim 1, wherein the circulatory assist pump is configured to operate wirelessly from within the subject's aorta.

9. The circulatory assist pump of claim 1, wherein the at least one impeller blade is configured to open such that a free end of the at least one impeller blade is substantially perpendicular to a central longitudinal axis of the impeller.

10. The circulatory assist pump of claim 1, wherein the at least one impeller blade comprises a plurality of impeller blades, each impeller blade of the plurality of impeller blades configured to open from a stowed position aligned with a central longitudinal axis of the impeller toward a proximal end of the circulatory assist pump and to a predetermined deployed state.

11. The circulatory assist pump of claim 10, wherein each impeller blade of the plurality of impeller blades exhibits a rounded shape, each impeller blade comprising a convex surface and a concave surface opposite the convex surface.

12. The circulatory assist pump of claim 11, wherein the convex surface of each impeller blade of the plurality of impeller blades, in the predetermined deployed state, is oriented axially toward a distal end of the circulatory assist pump.

13. A system for a circulatory assist pump, the system comprising:
a stent cage being expandable and collapsible, the stent cage of a size and shape to allow a highly open blood flow when the stent cage is positioned in an expanded state within a subject's aorta, the stent cage configured to be stable against an interior wall of the subject's aorta and flex with natural pulsatility of the subject's aorta when the stent cage is in the expanded state; and
at least one impeller encaged by the stent cage, each impeller comprising at least one impeller blade configured to selectively change shape.

14. The system of claim 13, wherein the at least one impeller blade comprises a shape memory material configured to change to a predetermined deployed state in response to the at least one impeller blade reaching a transition temperature of the shape memory material.

15. The system of claim 14, wherein the at least one impeller blade comprises a single helical impeller blade comprising a reinforced free end.

16. The system of claim 13, wherein the at least one impeller comprises multiple impellers arranged in series on a common driveline, each impeller encaged by the stent cage, and each impeller comprising impeller blades configured to selectively change shape.

17. The system of claim 16, the multiple impellers comprising a first impeller and a second impeller rotationally offset from the first impeller such that the impeller blades of the second impeller in a deployed state are rotationally offset from the impeller blades of the first impeller in the deployed state.

18. The system of claim 13, wherein the at least one impeller comprises impeller blades and a casing defining pockets corresponding to shapes of the impeller blades of the at least one impeller, the impeller blades configured to be received within the pockets.

19. A system for a circulatory assist pump, the system comprising:
a stent cage being expandable and collapsible, the stent cage of a size and shape to allow a highly open blood flow when the stent cage is in an expanded state within a subject's aorta, the stent cage further configured to provide a radial force against an inner wall of the subject's aorta when the stent cage is in the expanded state such that the stent cage is positionally secured to the inner wall of the subject's aorta and such that the stent cage flexes with a natural pulsatility of the subject's aorta;
an impeller encaged by the stent cage, the impeller comprising at least one impeller blade configured to selectively change shape; and
an insertion and removal catheter comprising an outer sheath configured to move axially relative to the stent cage to collapse the stent cage and the impeller within the outer sheath.

20. The system of claim 19, wherein the stent cage and the at least one impeller blade of the impeller are configured to expand to a deployed state when the outer sheath is axially retracted relative to the stent cage and the impeller.
